(19) United States
(12) Patent Application Publication
(10) Pub. No.: US 2025/0191772 A1
(43) Pub. Date: Jun. 12, 2025

(54) METHODS AND PRODUCTS FOR PREDICTING CANCER THERAPY-RELATED CARDIAC DYSFUNCTION

(71) Applicant: University Health Network, Toronto (CA)

(72) Inventors: Jason Edward FISH, Burlington (CA); Dakota Drew GUSTAFSON, Kingston (CA); Paaladinesh THAVENDIRANATHAN, Scarborough (CA); Christopher James MCINTOSH, Toronto (CA)

(21) Appl. No.: 18/951,068

(22) Filed: Nov. 18, 2024

Related U.S. Application Data
(60) Provisional application No. 63/600,554, filed on Nov. 17, 2023.

(51) Int. Cl. G16H 50/30; C12Q 1/6886; G01N 33/574; G01N 33/68; G16B 25/10; G16B 40/20

(52) U.S. Cl. CPC G16H 50/30; C12Q 1/6886; G01N 33/57488; G01N 33/6863; G16B 25/10; G16B 40/20; C12Q 2600/106; C12Q 2600/178

(57) ABSTRACT

A method of determining a likelihood of developing cancer therapy-related cardiac dysfunction (CTRCD) in a subject receiving an anthracycline and/or HER2 targeted therapy cancer treatment. Also disclosed herein is a method for treating a subject who is identified as being at risk of developing CTRCD. This disclosure also relates to a method of identifying if a subject with cancer to be treated with or an anthracycline and/or a HER2 targeted therapy treatment is likely to benefit from a cardioprotective treatment. Further disclosed is a panel or kit, the panel or kit comprising a plurality of detection agents specific for each of a set of biomarkers in a sample obtained from a subject with cancer, optionally selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α, and optionally a solid support.

### CROSS-REFERENCE TO RELATED APPLICATIONS

This application claims priority from U.S. Provisional Patent Application No. 63/600,554 filed Nov. 17, 2023, which is incorporated herein in its entirety by reference.

### FIELD

This disclosure relates to methods and products for predicting cardiac dysfunction in a subject that is or will be receiving anthracycline and/or HER2 targeted therapy cancer treatment. In particular, this disclosure relates to methods for predicting cancer therapy-related cardiac dysfunction (CTRCD) in a subject by measuring s set of biomarkers alone or in combination with other parameters. The disclosure also relates to a panel of biomarker detection agents for identifying risk of CTRCD.

### INTRODUCTION

Breast cancer remains not only the most diagnosed cancer in women but also one of the leading causes of cancer-related mortality¹. While optimized treatment regimens have increased overall survival of breast cancer patients, they also contribute to the development of heart failure (HF) which in turn is a competing risk for mortality amongst survivors²⁻⁹. In particular, women with human epidermal growth factor receptor 2 (HER2⁺) breast cancer receiving sequential therapy with anthracyclines and trastuzumab are at high risk of cancer therapy-related cardiac dysfunction (CTRCD) and HF^{2,10}. Identification of patients at risk of CTRCD prior to or early during cancer therapy remains an unmet clinical need^{11,12}.

While compromised cardiac function is indicative of CTRCD, observation of such changes may only occur after considerable myocardial damage has already occurred, potentially leading to irreversibility and heightened risk of mortality^{13,14}. Several blood biomarkers have been considered for risk assessment. Prior investigations of cardiac-centric blood-based biomarkers, including cardiac troponins (cTn), natriuretic peptides, as well as cardiac stress- or inflammatory-response proteins such as growth-differentiation factor-15 (GDF-15) have been conflicting⁵⁻²⁰. To date, there are limited data on biomarkers related to the vascular endothelium for risk prediction, despite the growing appreciation of the deleterious effects of antineoplastics on the vascular endothelium²¹⁻²⁸. For example, there is evidence of direct damage to the coronary microcirculation^{29,30} or induction of inflammation and coagulation^{24,31,32}, which indirectly impact endothelial health. Recent analyses of select endothelium-related markers such as nicotinamide adenine dinucleotide phosphate oxidase 4³³ or asymmetric dimethylarginine²⁶, or activation of coagulation or inflammatory markers^{24,34} have highlighted their involvement in CTRCD and their potential role in prognostication. In this respect, mechanisms in addition to direct cardiomyocyte damage, such as the induction of inflammation and endothelial dysfunction may be key driving factors of CTRCD³⁵.

Identifying those at risk of CTRCD prior to, or early during cancer treatment, is desirable.

Because of their stability in biosamples and high-dimensionality, transcriptomic profiling of the repertoire of circulating miRNAs in plasma has shown significant promise in providing not only detailed prognostic information, but also mechanistic insight, through their known roles in post-transcriptional gene regulation³⁶⁻³⁹. Recently, multidimensional approaches including utilization of machine learning models to integrate clinical data with broad omics technologies have revealed new avenues for efficiently delineating complex patient phenotypes and their associations with clinical outcomes⁴⁰⁻⁴³.

### SUMMARY

As demonstrated herein, the present inventors have leveraged a highly phenotyped cohort of women with early-stage HER2⁺ breast cancer who underwent thorough cardiovascular surveillance to: 1) assess the pre-therapy and temporal relationship between circulating measurements of inflammatory, cardiac-, and endothelial-centric protein marker expression analysis and whole transcriptome plasma miRNA sequencing with the subsequent development of CTRCD; and 2) determine and compare the prognostic value of pre-therapy cardiac imaging parameters, patient clinical demographics, and circulating markers for subsequent CTRCD using machine learning approaches.

The present disclosure relates to the identification of biomarkers including Angiopoietin-2, Endothelin-1 and Endoglin that were elevated prior to and during cancer treatment, and another biomarker (i.e., E-Selectin) that was elevated during treatment in patients that went on to develop CTRCD. Additionally, there were significant elevations in inflammatory biomarkers (including Myeloperoxidase, Interferon gamma-induced protein-10 and Interferon-α) before treatment in patients that developed CTRCD. Interferon gamma-induced protein-10 was also elevated during treatment in patients that developed CTRCD. Assessment of plasma miRNAs prior to treatment revealed distinct miRNA signatures in patients who went on to develop CTRCD.

Accordingly, the present disclosure provides a method of determining a likelihood of developing cancer therapy-related cardiac dysfunction (CTRCD) in a subject receiving an anthracycline and/or HER2 targeted therapy cancer treatment, comprising:
a) detecting or measuring levels of each of a set of biomarkers in a sample obtained from the subject with cancer, the set of biomarkers comprising at least one of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2; ANGPT2 and ENG; ANGPT2 and ET-1; Endoglin and MPO; ANGPT2, MPO and Endoglin; or ANGPT2, MPO, Endoglin, ET-1;
b) identifying the subject as being at an increased or decreased risk of developing CTRCD based on the presence of or the measured level of the set of biomarkers.

Another aspect of the disclosure is a method for treating a subject who is identified as being at risk of developing cancer therapy-related cardiac dysfunction (CTRCD) comprising:
a) detecting or measuring levels of a set of biomarkers in a sample obtained from a subject with cancer, optionally selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α;
b) identifying the subject as being at risk of developing CTRCD based on the levels of the set of biomarkers; and
c) administering a cardioprotective therapy and/or modifying the cancer therapy.

A further aspect of the disclosure is a method of identifying if a subject with cancer to be treated with or an anthracycline and/or a HER2 targeted therapy treatment is likely to benefit from a cardioprotective treatment, the method comprising,
a) detecting or measuring levels of each of a set of biomarkers associated with endothelial activation/dysfunction and/or inflammation in a sample obtained from the subject with cancer, the set of biomarkers comprising at least one of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2; ANGPT2 and ENG; ANGPT2 and ET-1; Endoglin and MPO; ANGPT2, MPO and Endoglin; or ANGPT2, MPO, Endoglin, ET-1;
   wherein the subject is likely to benefit when the levels of the set of biomarkers is indicative of an increased risk of CTRCD.

A further aspect of the disclosure is a panel, the panel comprising a plurality of detection agents specific for each of a set of biomarkers in a sample obtained from a subject with cancer, optionally selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α, and optionally a solid support.

In an embodiment, the set of biomarkers comprises at least one of angiopoietin-2 (ANGPT2), Endoglin (ENG), E-selectin, endothelin-1 (ET-1) and myeloperoxidase (MPO).

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 shows circulating cardiac biomarkers do not distinguish patients that will develop cancer therapy-related cardiac dysfunction (CTRCD): A) Schematic of cardiac imaging and biospecimen collection from the EMBRACE-MRI study (n=136 patients). B) Left ventricular ejection fraction (LVEF) as assessed by cardiac MRI (CMR). 37 of the patients (‘CTRCD’) developed CTRCD during the study period, while 99 patients (‘no CTRCD’) did not. C) Circulating cardiac biomarkers were assessed in patients at TP1-3. High sensitivity Cardiac Troponin I and B-Type Natriuretic Peptide (BNP) were measured in all patients (n=37 ‘CTRCD’ and n=99 ‘no CTRCD), while Growth/Differentiation Factor-15 (GDF-15) was measured in n=27 ‘CTRCD’ and n=27 ‘no CTRCD’ patients. Asterisks below indicate significant changes in the cohort at that timepoint compared to TP1. Asterisks above indicate significant differences between the ‘CTRCD’ and ‘no CTRCD’ groups at the specified timepoint.

FIG. 2 shows circulating inflammatory and endothelial dysfunction biomarkers distinguish patients that will develop CTRCD at baseline and early during cancer therapy: A) Select circulating inflammatory markers (myeloperoxidase [MPO], Interferon-α, Interferon gamma-induced protein-10 [IP-10], interleukin-1β [IL-1β]) were assessed at TP1-3. n=27 ‘CTRCD’ and n=27 ‘no CTRCD’ for MPO and n=25 ‘CTRCD’ and n=54 ‘no CTRCD’ for Interferon-α, IP-10 and IL-1. Data related to additional inflammatory markers are shown in FIG. 7. B) Circulating endothelial-centric markers (Angiopoietin-2 [ANGPT2], soluble E-Selectin [sE-Selectin], Endothelin-1 and Endoglin) were assessed at TP1-3. n=37 ‘CTRCD’ and n=70 ‘no CTRCD’. For panels A and B, asterisks below indicate significant changes in the cohort at the timepoint compared to TP1, while asterisks above indicate significant differences between the ‘CTRCD’ and ‘no CTRCD’ groups at the specified timepoint.

FIG. 3 shows analysis of pre-treatment levels of inflammatory and endothelial dysfunction biomarkers distinguishes those at risk of CTRCD. A) Measurement of Angiopoietin-2 (ANGPT2), soluble E-Selectin (sE-Selectin), Endoglin, Endothelin-1 [ET-1], myeloperoxidase (MPO), high sensitivity cardiac Troponin I (hs-cTroponin I), b-type natriuretic protein (BNP) and growth/differentiation factor-15 (GDF-15) in all baseline samples (n=37 ‘CTRCD’ and n=99 ‘no CTRCD’). B) Correlation plots of the indicated analytes at TP1 in all baseline samples. A correlation matrix of all factors is shown in FIG. 8.

FIG. 4 shows plasma miRNAs are differentially expressed at baseline in patients that will develop CTRCD. Volcano plot indicates up- and down-regulated plasma miRNAs at TP1 and pathway analysis of miRNA targets in the entire cohort. Predicted concordant up-regulated (i.e., miRNAs down-regulated in CTRCD) and down-regulated (i.e., miRNAs up-regulated in CTRCD) KEGG pathways from differentially expressed miRNAs are indicated. Volcano plot data are displayed as false discovery rate-adjusted P values (Q values) vs. the log 2 fold change, with dashed lines drawn to indicate +/−1.5-fold difference. All miRNAs with a Q value <0.05 are highlighted.

FIG. 5 shows machine learning identifies features that predict CTRCD risk from baseline data. A) Assessment of datasets using leave-one-out cross-validation with a Random Forest model for various combinations of Clinical, CMR, Protein and miRNA data. Shown is the area under the receiver operating characteristic (AUROC) score, confidence intervals obtained via bootstrapping and specificity of the models. B) The average impact of variables grouped by type (Clinical, CMR, Protein, miRNA) on model output as determined by mean SHAP values. C) Contribution of the impact of individual variables on model output as determined by SHAP value. Red indicates high values for the variable and blue indicates low values. Protein, CMR, miRNA and Clinical variables are indicated using red, blue, orange and green text, respectively. D) The average impact of variables on model impact as measured by mean SHAP value.

FIG. 6 shows biomarker associations with heightened risk A) Plot indicating an approximation of how the model prediction of the event probability changes as a function of two protein features with white being zero and red being model max (100%). Overlaid is the actual patient data from the EMBRACE-MRI cohort with ‘no CTRCD’ in blue, and ‘CTRCD’ as red dots. Pairwise comparisons are ENG+ANGPT2, ANGPT2+MPO and ENG+MPO. B) Three-dimensional scatterplot of ANGPT2, MPO and ENG concentrations in all baseline samples. C) The contribution of the impact of circulating protein concentrations of angiopoietin-2 (ANGPT2), myeloperoxidase (MPO), endoglin (ENG) and endothelin-1 (ET-1) on the output of the full model as determined by plotting SHAP values for each concentration.

FIG. 7 shows profiling of circulating inflammatory and angiogenic markers at baseline and early stages of treatment: Several inflammatory markers were measured at baseline (i.e., TP1) and at early stages of treatment (i.e., TP2 and TP3): Eotaxin, Granulocyte colony-stimulating factor (G-CSF), Granulocyte-macrophage colony-stimulating factor (GM-CSF), Interferon-γ (IFN-γ), Interleukin-2 (IL-2), IL-8, IL-10, IL-15, IL-17A, Interleukin-1 receptor antagonist (IL-1 Ra), Monocyte chemoattractant protein-1 (MCP-1), Macrophage inflammatory protein-1α (MIP-1α), MIP-1β, Tumour necrosis factor-α (TNF-α). Angiogenic factors (Epidermal growth factor [EGF] and Vascular endothelial growth factor [VEGF]) were also measured. n=25 ‘CTRCD’ and n=54 ‘no CTRCD’. Although not shown, IL-1a, IL-3, IL-4, IL-5, IL-6, IL-7, IL-12p40, IL-12p70, IL-13 and TNF-3 were also measured, but values were undetectable in most samples so were excluded.

FIG. 8 shows correlations among circulating biomarkers measured at baseline. Correlation matrix of circulating biomarkers at baseline (i.e., TP1) in the full cohort is shown. (n=37 ‘CTRCD’ and n=99 ‘no CTRCD’).

FIG. 9 shows machine learning identifies variables that contribute to CTRCD risk prediction from baseline data: A) Assessment of datasets using leave-one-out cross-validation with a Random Forest model for CMR data alone. Shown is the contribution of the impact of individual variables (left and middle panels) and grouped variables (top right panel) on model output as determined by mean SHAP values. The AUROC score, confidence intervals and specificity of the model is shown in the bottom right panel. B) For the Clinical+CMR data model, the contribution of the impact of individual variables (left and middle panels) and grouped variables (right panel) on model output as determined by mean SHAP values is shown. C) For the Clinical+CMR+Protein data model, the contribution of the impact of individual variables (left and middle panels) and grouped variables (right panel) on model output as determined by mean SHAP values is shown. D) The AUROC curve for miRNA data only is shown. The score, confidence intervals and specificity of the model is shown. Labels for Clinical, CMR, Protein and miRNA variables are indicated in green, blue, red and orange text, respectively.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” or “approximately” the indicated value, and take into account experimental error and variations that would be expected by a person having ordinary skill in the art. Further, it is to be understood that “a,” “an,” and “the” include plural referents unless the content clearly dictates otherwise. The term “about” means plus or minus 0.1 to 20%, 5-20%, or 10-20%, preferably 5-15%, more preferably 5% or 10%, of the number to which reference is being made.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The phrase “at least one” when used herein in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Terms of degree such as “about”, “substantially”, and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “sample” as used herein refers to a sample of fluid or tissue sample derived or obtained from a subject. Examples of fluid samples include, but are not limited to, blood, plasma, serum, urine, spinal fluid, lymph fluid, tears, saliva, sputum and milk. Methods of obtaining such samples are known in the art including but not limited to standard blood retrieval procedures.

The term “plasma” or a derivative thereof as used herein refers to component of blood that holds the blood cells in whole blood in suspension. Plasma is the liquid part of the blood that carries cells and proteins throughout the body.

The term “serum” or a derivative thereof as used herein refers to the fluid and solute component of blood which does not play a role in clotting. Serum is blood plasma without fibrinogens.

The term “platelet poor plasma” or a derivative thereof as used herein refers to blood plasma with very low number of platelets, for example, <10×10³/μL). The skilled person in the art can readily identify known methods for collecting plasma and for preparing platelet poor plasma, for example, methods involving centrifugation that sediment platelet. Platelet poor plasma contain extracellular vesicles and it is useful for laboratory assays, including analysis of contents and characteristics of extracellular vesicles, including their sizes and abundance (i.e. concentration).

The terms “miRNA” or “microRNA” as used herein refer to short, single-stranded RNA molecules approximately 21-23 nucleotides in length which are partially complementary to one or more mRNA molecules (target mRNAs). MiRNAs down-regulate gene expression by inhibiting translation or by targeting the mRNA for degradation or deadenylation. MiRNAs base-pair with miRNA recognition elements (MREs) located on their mRNA targets, usually on the 3′-UTR, through a region called the ‘seed region’ which includes nucleotides 2-8 from the 5′-end of the miRNA. Matches between a miRNA and its target are generally asymmetrical. The complementarity of seven or more bases to the 5′-end miRNA has been found to be sufficient for regulation.

MiRNAs are first transcribed as primary transcripts (pri-miRNA) by RNA polymerase II or RNA polymerase III. Generally, a pri-miRNA comprises a double stranded stem of about 33 base pairs, a terminal loop and two flanking unstructured single-stranded segments. Pri-miRNA is processed by a protein complex which consists of an RNase III enzyme (Drosha), and a double stranded-RNA binding protein (DGCR8 or DiGeorge syndrome critical region 8 gene) resulting in a short 70-nucleotide stem-loop structure called pre-miRNA. The pre-miRNA is transported from the nucleus to the cytoplasm by Exportin-5 (Exp-5) by the action of RanGTPase. In the cytoplasm, Dicer (an RNAse III endonuclease) cleaves the pre-miRNAs into short RNA duplexes termed miRNA duplexes. After cleavage, the miRNA duplex is unwound by an RNA helicase and the mature miRNA strand binds to its target mRNAs, and the complementary strand (i.e. passenger strand) is degraded.

The term “subject” as used herein includes all members of the animal kingdom, for example human.

The term “cancer therapy-related cardiac dysfunction” or “CTRCD” as used herein refers to a condition that can be defined as a decrease in left ventricular ejection fraction (LVEF) of more than 5% to below the lower limit of normal, which is considered an LVEF of 55%, with cardiac symptoms, or if these definitions were not met, then a decrease greater than 10% in global longitudinal strain (GLS) compared with a baseline GLS was considered subclinical left ventricular (LV) dysfunction. CTRCD can be classified as direct (dose-dependent vs dose-independent) or indirect.

As used herein, the term “treating” includes abrogating, substantially inhibiting, slowing or reversing the progression of a condition, such as slowing or reversing progression, substantially ameliorating clinical or aesthetical symptoms of a condition such as improving or substantially preventing the appearance of clinical or aesthetical symptoms of a condition such as CRTCD. Treatment can include prophylactic treatment, for example before clinical signs of CRTCD.

As used herein, the term “administration” or a derivative thereof means to provide or give a subject an agent optionally a treatment, such as a composition comprising an effective amount of an anti-neoplastic agent such as an anthracycline and/or a HER2 targeted therapy by an effective route.

The term “cancer” as used herein refers to one of a group of diseases caused by the uncontrolled, abnormal growth of cells that can spread to adjoining tissues or other parts of the body. Cancer cells can form a solid tumor, in which the cancer cells are massed together, or exist as dispersed cells. The cancer may be of any stage (early stage, locally advanced, or advanced), and may optionally be metastatic cancer, relapsed cancer, refractory cancer, and/or cancer with acquired chemoresistance. In an embodiment, the cancer is a HER2+ cancer. A HER2+ cancer is a cancer that has extra copies of the HER2 gene or over-expression of said gene. Cancer types include, but are not limited to, breast cancer, hematological cancers such as leukemia, lymphoma and myeloma, thymoma, sarcomas, stomach cancer, uterine cancer, ovarian cancer, renal cancer, lung cancer and melanoma.

The term “HER2 targeted therapy” as used herein refers to tyrosine kinase inhibitors or monoclonal antibodies that bind and inhibit HER2. These compounds are useful for treating cancers, including breast cancer, gastrointestinal cancers, ovarian cancer, colorectal cancer, esophageal cancer, lung cancer and stomach cancer. For example, HER2 targeted therapies include trastuzumab, pertuzumab, margetuximab, ado-trastuzumab emtansine, trastuzumab deruzumab deruxtecan, tucatinib, neratinib, lapatinib and their derivatives.

The term “cancer treatment” or “cancer therapy” or a derivative thereof as used herein refers to a therapeutic agent that are useful for treating cancer. An anti-cancer agent can be a drug such as a small molecule drug, a biologic such as a polypeptide, a therapeutic protein, an antigen, an antibody, or an antigen binding fragment. The antibody can be a monoclonal, polyclonal, chimeric, humanized antibody, or a fragment thereof, or a combination thereof. For example, trastuzumab (also known as Herceptin) is a humanized monoclonal antibody that is an anti-neoplastic agent. The antigen binding fragment is a Fab, Fab′, F(ab′)2, scFv, dsFv, ds-scFv, dimer, minibody, diabody, or multimer thereof or bispecific antibody fragment, or a combination thereof. An anti-neoplastic agent (e.g. cancer treatment) can be a chemotherapeutic agent or an immunotherapeutic agent. An anti-neoplastic agent may be used alone or in combination with another anti-neoplastic agent, or in combination with any other medication or modality.

The term “anthracycline” as used herein refers to a class of drugs used in cancer chemotherapy that are extracted from Streptomyces bacterium. These compounds are useful for treating cancers, including leukemia, lymphoma, breast cancer, stomach cancer, uterine cancer, ovarian cancer, bladder cancer, and lung cancers. Examples of anthracyclines include doxorubicin, daunorubicin, epirubicin and idarubicin, and their derivatives and analogues.

The term “control” as used herein refers to a sample taken from a subject or a group of subjects who are either known as having a particular condition or trait or as not having a particular condition or trait. The control can vary depending on what is being monitored, assessed or diagnosed. For example, a cancer-free subject, a cancerous subject which developed CTRCD, a cancerous subject which did not develop CTRCD, a sample from the same subject prior to treatment or a specific value or dataset that can be used to prognose or classify the value e.g., risk of developing CTRCD. The control can also be a predetermined standard or reference range of values, determined from a group of subjects for example as described herein. The control can be a cut-off value, above or below which the subject, is at increased risk. The skilled person will be able to adjust the standard or reference range.

The term, “cardioprotective therapy” as used herein refers to agents that can prevent or treat cardiotoxicity, for example, CTRCD. Cardioprotective therapies include, for example, dexrazoxane, dantrolene, statins and SGLT2 inhibitors.

It should also be understood that, in certain methods described herein that include more than one step or act, the order of the steps or acts of the method is not necessarily limited to the order in which the steps or acts of the method are recited unless the context indicates otherwise.

Although any methods and materials similar or equivalent to those described herein can also be used in the practice or testing of the present disclosure, examples of methods and materials are now described.

### II. Methods and Materials

As described herein, is a method of determining a likelihood of developing cancer therapy-related cardiac dysfunction (CTRCD) in a subject receiving an anthracycline and/or HER2 targeted therapy cancer treatment, comprising:
a) detecting or measuring levels of each of a set of biomarkers in a sample obtained from the subject with cancer, the set of biomarkers comprising at least one of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2; ANGPT2 and ENG; ANGPT2 and ET-1; Endoglin and MPO; ANGPT2, MPO and Endoglin; or ANGPT2, MPO, Endoglin, ET-1;
b) identifying the subject as being at an increased or decreased risk of developing CTRCD based on the presence of or the measured level of the set of biomarkers.

Also provided herein, for treating a subject who is identified as being at risk of developing cancer therapy-related cardiac dysfunction (CTRCD) comprising:
a) detecting or measuring levels of a set of biomarkers in a sample obtained from a subject with cancer, optionally selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α;
b) identifying the subject as being at risk of developing CTRCD based on the levels of the set of biomarkers; and
c) administering a cardioprotective therapy and/or modifying the cancer therapy.

Also provided herein, is a method of identifying if a subject with cancer to be treated with or an anthracycline and/or a HER2 targeted therapy treatment is likely to benefit from a cardioprotective treatment, the method comprising,
a) detecting or measuring levels of each of a set of biomarkers associated with endothelial activation/dysfunction and/or inflammation in a sample obtained from the subject with cancer, the set of biomarkers comprising at least one of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2; ANGPT2 and ENG; ANGPT2 and ET-1; Endoglin and MPO; ANGPT2, MPO and Endoglin; or ANGPT2, MPO, Endoglin, ET-1;
   wherein the subject is likely to benefit when the levels of the set of biomarkers is indicative of an increased risk of CTRCD.

Further provided is a panel, the panel comprising a plurality of detection agents specific for each of a set of biomarkers in a sample obtained from a subject with cancer, optionally selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α, and optionally a solid support. The panel can include other biomarkers described herein.

As used herein, “angiopoietin-2” or “ANGPT2” refers to a growth factor in the angiopoietin family. The nucleotide and amino acid sequence of human ANGPT2 can be found at, for example, GenBank Accession No. AB009865.1 or UniProt ID: 015123.

As used herein, “Endoglin” or “ENG”, refers to a vascular endothelium glycoprotein which plays a role in the regulation of angiogenesis. The nucleotide and amino acid sequence of human ENG can be found at, for example, GenBank Accession No. AH006911.2 or UniProt ID: P17813.

As used herein, “E-selectin”, refers to a cell-surface glycoprotein which plays a role in immunoadhesion. The nucleotide and amino acid sequence of human E-selectin can be found at, for example, GenBank Accession No. M30640.1 or UniProt ID: P16581.

As used herein, “endothelin-1” or “ET-1” refers to an endothelium-derived vasoconstrictor peptide. The nucleotide and amino acid sequence of human ET-1 can be found at, for example, GenBank Accession No. J05008.1 or UniProt ID: P05305.

As used herein, “myeloperoxidase” or “MPO” refers to an enzyme that catalyzes the production of hypohalous acids and is part of the host defense system of polymorphonuclear leukocytes. The nucleotide and amino acid sequence of human MPO can be found at, for example, GenBank Accession No. J02694.1 or UniProt ID: P05164.

As used herein, “interferon gamma-induced protein-10” or “IP-10” or “CXCL10” refers to a pro-inflammatory cytokine. The nucleotide and amino acid sequence of human IP-10 can be found at, for example, GenBank Accession No. X02530.1 or UniProt ID: P02778.

As used herein, “Interferon-α” or “IFN” encompasses the interferon-α subtypes, for example including but not limited to, IFN-α1, IFN-α2, IFN-α4, IFN-α10, IFN-α21. The nucleotide and amino acid sequence of human IFN-α4 can be found for example, at GenBank Accession No. M27318.1 or UniProt ID: P05014.

The level of a protein can be detected or measured by a technique known in the art, including proteome profiling techniques such as liquid chromatograph and/or mass spectrometry including nanoscale liquid chromatography coupled to tandem mass spectrometry (nano-LC-MS/MS). In some embodiments, detecting a presence of or measuring a level of one or more of proteins, optionally cardiac proteins, or circulating protein biomarker, optionally an inflammatory marker, optionally a pro-inflammatory cytokine comprises using proteome profiling, optionally liquid chromatograph and/or mass spectrometry, optionally nanoscale liquid chromatography coupled to tandem mass spectrometry (nano-LC-MS/MS).

In an embodiment, the set comprises or comprises at least one biomarker. In an embodiment, the set comprises or comprises at least two biomarkers. In an embodiment, the set comprises or comprises at least three biomarkers. In an embodiment, the set comprises or comprises at least four biomarkers. In other embodiments, the set comprises or comprises at least five biomarkers. The set can comprise any combination of biomarkers described herein.

In an embodiment, the set of biomarkers comprises ANGPT2 and/or ENG.

In an embodiment, the set of biomarkers comprises E-selectin.

In an embodiment, the set of biomarkers comprises ET-1.

In an embodiment, the set of biomarkers is MPO.

In an embodiment, the set of biomarkers comprises IP-10 or interferon-α.

In an embodiment, the set of biomarkers are MPO, ANGPT2 and ENG.

In an embodiment, the one or more biomarkers are MPO, ANGPT2 and ENG.

In an embodiment, the set of biomarkers comprises or consists of MPO, ANGPT2, ENG and ET-1.

In an embodiment, the set of biomarkers comprises or consists of MPO, ANGPT2, ET1, ENG, E-Selectin and GDF-15.

In an embodiment, the cancer is selected from a breast cancer, thymoma, sarcoma, a gastrointestinal cancer, breast cancer, a hematological cancer, lung cancer, renal cancer or melanoma.

In an embodiment, the cancer is breast cancer.

In an embodiment, the breast cancer is human epidermal growth factor 2 positive (HER2+) breast cancer.

In an embodiment, the HER2+ breast cancer is stage I, II, or III HER2+ breast cancer.

In an embodiment, the sample obtained from the subject is obtained prior to starting the cancer treatment.

In an embodiment, the set of biomarkers measured or detected is ANGPT2, ENG, ET-1 and MPO, wherein an increased level in the sample obtained prior to administration compared to a control (e.g. non-CTRCD) is indicative of an increased risk of developing CTRCD.

In an embodiment, the subject has initiated the cancer treatment.

In an embodiment the sample is obtained early in the cancer treatment, optionally, within 6 months of diagnosis or initiation of the cancer treatment.

In an embodiment, the set of biomarkers measured or detected comprises ANGPT2 and ENG, wherein an increased level in the sample obtained early in the cancer treatment compared to a control (e.g., pre-treatment control) is indicative of an increased risk of developing CTRCD.

In an embodiment, wherein the set of biomarkers measured or detected is E-selectin and ET-1, wherein an increased level in the sample obtained early in the cancer treatment compared to a control (e.g., pre-treatment control) is indicative of developing CTRCD.

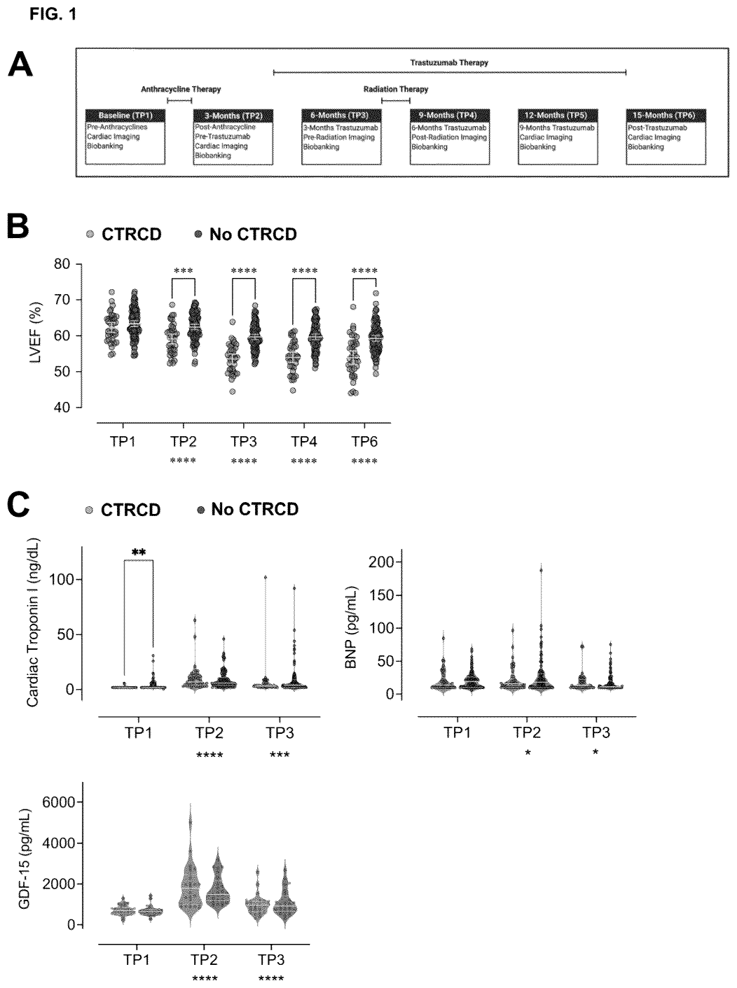

The cancer treatment can be broken up into timepoints (FIG. 1A). For example, prior to treatment is time-point 1 [TP1]. Early cancer treatment includes 3 months (post-anthracycline) referred to as time-point 2 [TP2] and 6 months (3 months trastuzumab) referred to as time-point 3 [TP3]. Other time-points include 9 months (6 months trastuzumab/post-radiation) referred to as time-point 4 [TP4], 12 months (9 months trastuzumab) referred to as time-point 5 [TP5] and 15 months (post-trastuzumab) referred to as time-point 6 [TP6]. The sample can be taken during any of these timepoints. In an embodiment, the sample is taken prior to the cancer treatment, for example TP1. In an embodiment, the sample is taken early in the cancer treatment, for example TP2 and/or TP3. In an embodiment, the sample is taken at TP4, TP5 and/or TP6. In an embodiment, the sample is taken at after the cancer treatment, for example beyond TP6.

In a further embodiment, one or more sample are taken at one or more time points. For example, a sample is taken prior to the cancer treatment and one or more subsequent samples is taken during treatment and/or after the cancer treatment. In an embodiment, subsequent samples can be obtained and compared. In an embodiment, a subject can be monitored for the development, progression or amelioration of CTRCD. The sample can also be taken after initiating a cardioprotective treatment and the method can be used for monitoring response to the cardioprotective treatment. For example, a subject receiving a cancer treatment and subsequently being identified and or treated with a cardioprotective treatment could be monitored to assess whether the CTRCD is improving, not improving or worsening. If for example, the subject shows improvement or worsening, the cardioprotective treatment, and/or the cancer treatment could be altered.

In an embodiment, the cancer treatment is an anthracycline and a Her-2 specific monoclonal antibody, optionally a humanized monoclonal antibody.

In an embodiment, the anthracycline is selected from daunorubicin, doxorubicin, epirubicin, idarubicin, mitoxantrone or valrubicin or an analogue of any thereof or combinations of any thereof.

In an embodiment, the anthracycline is doxorubicin or a doxorubicin analogue optionally daunorubicin, epirubicin and idarubicin.

In an embodiment, the HER2 targeted therapy comprises trastuzumab, pertuzumab, or tucatinib or analogs of any thereof or combinations of any thereof.

In an embodiment, the HER2 targeted therapy is or comprises trastuzumab.

In an embodiment, the sample was obtained from the subject within 3 months of initiating the HER2 targeted therapy treatment.

In an embodiment, the cancer treatment further comprises radiation therapy.

In an embodiment, the sample was obtained from the subject after starting a first dose of a chemotherapeutic agent.

In an embodiment, the sample was obtained after initiation of anthracycline treatment and the set of biomarkers comprises ANGPT2, E-Selectin and Endoglin.

In an embodiment, the sample was obtained after initiation of HER2 targeted therapy and the set of biomarkers comprises ANGPT2, E-Selectin, Endoglin and ET-1.

In an embodiment, the set of biomarkers comprises at least one of angiopoietin-2 (ANGPT2), Endoglin (ENG), E-selectin, endothelin-1 (ET-1) and myeloperoxidase (MPO).

In an embodiment, the set of biomarkers comprises at least two of angiopoietin-2 (ANGPT2), Endoglin (ENG), E-selectin, endothelin-1 (ET-1) and myeloperoxidase (MPO).

In an embodiment, the set of biomarkers comprises at least three of angiopoietin-2 (ANGPT2), Endoglin (ENG), E-selectin, endothelin-1 (ET-1) and myeloperoxidase (MPO).

In an embodiment, the set of biomarkers comprises at least four of angiopoietin-2 (ANGPT2), Endoglin (ENG), E-selectin, endothelin-1 (ET-1) and myeloperoxidase (MPO).

In an embodiment, the set of biomarkers comprises angiopoietin-2 (ANGPT2), Endoglin (ENG), E-selectin, endothelin-1 (ET-1) and myeloperoxidase (MPO).

In an embodiment, the set of biomarkers comprises or consists of at least four of MPO, ANGPT2, ET1, ENG, E-Selectin and GDF-15.

In an embodiment, the levels of the set of biomarkers is measured using a panel or kit comprising detection agents for each of the set of biomarkers.

Any immunoassay or immunocytochemistry format can be used. For example, polypeptide biomarkers can be measured or detected by ELISA, western, or other immunoassay. The ELISAs can use various modes of detection, including colorimetric, fluorescence, fluorescence polarization, time-resolved fluorescence, luminescence and chemiluminescence. Surface plasmon resonance binding assays can also be used.

The detection agents can be antibodies specific for a particular biomarker.

In an embodiment, the method or panel further comprises detecting a presence of or measuring a level of one or more miRNA.

In an embodiment, the one or more miRNAs comprises miR-6727-5P and/or miR-1915-3P.

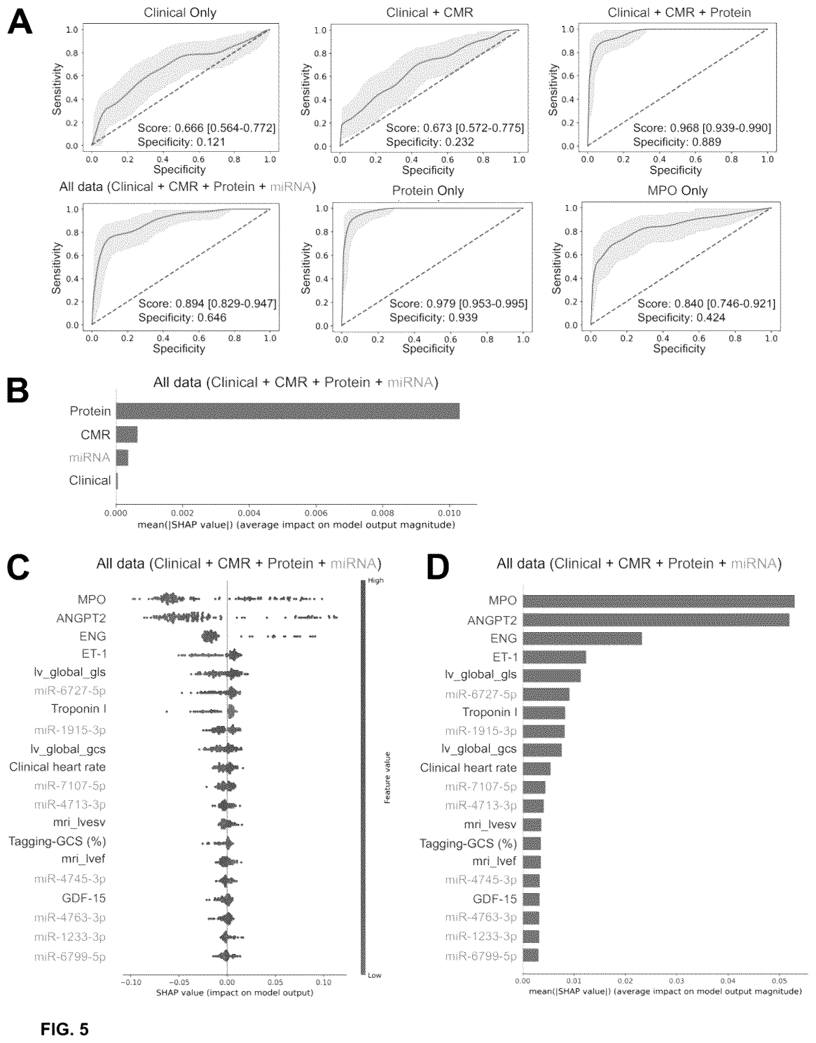

In a further embodiment, the one or more miRNAs comprise any of the features in FIG. 5.

The presence or level of miRNA in a sample can be detected using high-throughput whole miRNA transcriptome sequencing techniques. In an embodiment, detecting a presence of or measuring a level of one or more of miRNAs associated with angiogenesis and/or oxidative stress comprises using whole miRNA transcriptome sequencing techniques.

The level of miRNA in a sample can also be detected using PCR based methods or with one or more hybridization probes. In an embodiment, measuring the level of miRNA comprises using microarray analysis. In another embodiment, a quantitative PCR assay is used to measure the level of miRNA. In yet a further embodiment, the level of miRNA is detected by isolating miRNA, and hybridized said miRNA with a hybridization probe, for example an antisense molecule described herein.

In another embodiment, measuring the level of miRNA comprises (a) polyadenylating the miRNA with ATP and a poly(A) polymerase to form a polyadenylated miRNA having a sequence of contiguous A residues; (b) reverse transcribing the polyadenylated miRNA to form a cDNA in a reaction mixture comprising (i) a first primer of not more than 40 nucleotides in length having complementarity to at least two 3′ terminal nucleotides of the miRNA and the sequence of contiguous A residues of the polyadenylated miRNA so as to hybridize therewith and initiate synthesis of a cDNA complementary to the polyadenylated miRNA, (ii) a reverse transcriptase and (iii) all four deoxyribonucleoside triphosphates; (c) amplifying a DNA molecule comprising the cDNA in a reaction mixture comprising (i) the cDNA, (ii) the first primer; (iii) a second primer that is sufficiently complementary to the 3′ nucleotides of the cDNA to hybridize therewith and initiate synthesis of an extension product; (iv) a DNA polymerase and (v) all four deoxyribonucleoside triphosphates; and (d) detecting and/or quantifying the amplified DNA molecule, wherein the presence and/or quantity of the amplified DNA corresponds to that of the miRNA. In a further embodiment, the primer used to during the measuring of miRNA levels is designed using the methods described in Balcells et al., 2018. In another embodiment, detecting and/or quantifying the amplified cDNA molecule comprises utilizing real time RT-PCR. In a further embodiment, detecting and/or quantifying the amplified cDNA comprises utilizing gel electrophoresis.

In an embodiment, the method or panel further comprises one or more clinical parameter identified in FIG. 5.

In a further embodiment, the method or panel further comprises obtaining cardiac clinical data and/or imaging data, optionally the imaging data comprises one or more of cardiac magnetic resonance imaging (MRI) and echocardiography, optionally the MRI is left ventricular global longitudinal and circumferential strain.

The method described herein can further include obtaining cardiac clinical data or parameters, which may include cardiac imaging data such as echocardiography and cardiac magnetic resonance imaging (MRI). These cardiac clinical data may inform on cardiac dysfunction in a subject. In some embodiments, the method further comprises obtaining left ventricular global longitudinal and circumferential strain, optionally the cardiac clinical data comprises cardiac imaging data, optionally the cardiac imaging data comprises one or more of echocardiography and at least one cardiac magnetic resonance imaging (MRI) variable. The cardiac MRI variables include 3-dimensional and cardiac MRI left ventricular shape and function, global longitudinal strain, myocardial T1 and T2 mapping, and extracellular volume fraction. In some embodiments, the at least one cardiac MRI variable comprises one or more of 3-dimensional and cardiac MRI left ventricular shape and function, global longitudinal strain, myocardial T1 and T2 mapping, and extracellular volume fraction.

In an embodiment, the panel or method further comprises one or more clinical variable as identified in Supplemental Table III.

In another embodiment the identifying step comprises comparing the measured level of the one or more biomarkers, the measured level of one or more miRNAs, the cardiac clinical data and/or imaging data, and/or the one or more clinical variables to a reference level, profile or score, optionally via a trained classifier.

In an embodiment, the sample is a blood sample.

In an embodiment, the blood sample is plasma or serum fraction.

In a further embodiment, the method or panel further comprises determining whether the subject has hyperlipidemia.

In an embodiment, the panel is a plate or bead, optionally the panel is an enzyme-linked immunosorbent assay (ELISA).

In an embodiment, the panel is for detecting cancer therapy-related cardiac dysfunction (CTRCD) risk in a subject.

In an embodiment, the panel is for use in a method as described herein.

In an embodiment, the cardioprotective therapy includes dexrazoxane, dantrolene statins and SGLT2 inhibitors.

In an embodiment, wherein a subject is identified as being at risk of developing cancer therapy-related cardiac dysfunction (CTRCD), the cardioprotective therapy is administered prior to cancer treatment.

In an embodiment, the cardioprotective therapy is administered during cancer treatment.

In an embodiment, the cardioprotective therapy is administered after cancer treatment.

In an embodiment, the method further comprises assessing if the cardioprotective treatment is effective. For example, the subject can be monitored for one or more of the biomarkers described herein to assess if the treatment is effective.

Machine learning methods are useful for informing cardiac dysfunction that takes into account the presence or measured level of biomarkers, miRNAs, the cardiac clinical data, and/or clinical variables and/or by comparing the presence or measured level of biomarkers, miRNAs, the cardiac clinical data, and/or clinical variables, to a reference level, profile or score. A classifier is a discrete-valued function that is used to assign class labels to particular data points. A classifier utilizes training data to understand how given input variables relate to the class. When the classifier is trained, it can be used to predict an outcome such as cardiac dysfunction, thus, trained classifiers can be used to inform on cardiac dysfunction and/or reference level, profile or score of cardiac function. Utilization of biomarkers described herein, and optionally miRNAs, clinical parameters, cardiac clinical data, clinical variables and machine learning algorithms are useful for grouping patients into those who will not experience cardiotoxic responses and those who will experience cardiotoxic responses, as shown in the Examples.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure:

### Example 1: Methods

Synopsis of Study Design and Participant Demographics: The primary results were generated through a secondary analysis of a prospectively recruited single-center longitudinal cohort study enrolling 136 consecutive patients with early-stage (Stages I-) HER2⁺ breast adenocarcinoma receiving sequential therapy with anthracyclines and trastuzumab with or without adjuvant radiotherapy (Evaluation of Myocardial Changes During BReast Adenocarcinoma Therapy to Detect Cardiotoxicity Earlier With MRI [EMBRACE-MRI]1; NCT02306538) between November 2013 and July 2019 (Table I, Supplemental Table I, and FIG. 1A)⁴⁴. Of these participants, 37 (27.2%) had a final diagnosis of CTRCD on the basis of the definitions provided by the ‘Cardiac Review and Evaluation Committee’ (CREC; see below for clinical definitions)⁴⁵. All participants were 18 years of age or older and provided written informed consent in accordance with protocols approved by the REB of the University Health Network (REB #19-5719.0). The study and all associated protocols abide by the ethical principles for medical research set forth by the Helsinki Declaration II⁴⁶. All patients had baseline cardiac magnetic resonance imaging (CMR) and two-dimensional echocardiography (2DE) performed at baseline (prior to treatment, time-point 1 [TP1]), as well as at several timepoints during cancer treatment: 3 months (post-anthracycline, TP2), 6 months (3 months trastuzumab, TP3), 9 months (6 months trastuzumab/post-radiation, TP4), 12 months (9 months trastuzumab, TP5) and 15 months (post-trastuzumab, TP6) (FIG. 1A). Blood samples were collected and biobanked at all time-points.

TABLE I
Characteristics of the Participants at Baseline. *

| | No CTRCD | CTRCD |
|---|---|---|
| Characteristic ^{†} | (N = 76) | (N = 36) |
| Age | | |
| Median (range)-yr | 51 (28-68) | 53 (35-70) |
| Distribution-no. (%) | | |
| 18-40 yr | 12 (15.8) | 5 (13.9) |
| 41-64 yr | 62 (81.6) | 27 (75.0) |
| ≥65 | 2 (2.6) | 4 (11.1) |
| Body mass index-kg/m^{2 ‡} | 25.6 ± 4.5 | 26.2 ± 5.9 |
| CV risk factor-no. (%) | | |
| Hypertension | 12 (15.8) | 4 (11.1) |
| Hyperlipidemia | 6 (7.9) | 5 (13.9) |
| Diabetes | 2 (2.6) | 2 (5.6) |
| Smoker ^{§} | 17 (22.4) | 11 (30.6) |
| Coronary Artery Disease | 0 (0.0) | 0 (0.0) |
| Congestive Heart Failure | 0 (0.0) | 0 (0.0) |
| Blood pressure (mmHg) | | |
| Systolic | 115.7 ± 14.3 | 116.6 ± 15.8 |
| Diastolic | 73.2 ± 8.6 | 75.8 ± 11.2 |
| Cardiac laboratory testing | | |
| Troponin (ng/dl) ^{¶, ∥} | 2.2 ± 1.7 | 2.1 ± 0.5 |
| B-type Natriuretic Peptide (pg/mL) ^{¶} | 20.6 ± 12.3 | 20.0 ± 16.0 |
| Hematocrit (%) | 40.3 ± 3.0 | 40.3 ± 3.2 |
| Cardiovascular therapy-no. (%) | | |
| Angiotensin-converting enzyme | 1 (1.3) | 4 (11.1) |
| inhibitors | | |
| Angiotensin receptor blockers | 6 (8.6) | 0 (0.0) |
| Beta-blockers | 5 (6.6) | 0 (0.0) |
| Statins | 4 (5.2) | 2 (5.6) |
| Metastatic disease-no./total no. (%) | | |
| Radiation-no./total no. (average rads) | | |
| Left breast | 35/76 (191) | 21/36 (211) |
| Right breast | 35/76 (100) | 15/36 (95) |
| Heart ** | 65/72 (168.1) | 32/34 (179.4) |
| Therapeutics-no./total no. | | |
| (average dose mg) | | |
| Epirubicin | 3/76 (403) | 2/35 (424) |
| Doxorubicin ^{∥} | 73/76 (515) | 33/35 (518) |
| Trastuzumab ^{∥} | 76/76 (6889) | 35/35 (7121) |

* The summary statistics are based off on the full population indicated in the column heading.
^{†} Plus-minus values are means ± SD.
^{‡} Body mass index is the weight in kilograms divided by the square of the height in meters.
^{§} Smoker is defined as an aggregate of self-reported current and previous smokers.
^{¶} Values below the laboratory diagnostic reference ranges were converted to the minimally reportable value of (2 ng/dL for troponin and 10 pg/mL for BNP).
^{∥} Data were not available for 1 patient in the CTRCD group.
** Data were not available for 4 patients in the no CTRCD group and 2 patients in the CTRCD group.
There were no significant differences (p < 0.05) between no CTRCD and CTRCD groups based on t-test for continuous variables, Fischer's exact test for binary/categorical variables, and Wilcoxon rank sum test for therapeutic dosages.

Supplemental Table I: Inclusion and exclusion criteria for EMBRACE-MRI study

Participant Selection
Inclusion Criteria

| | |
|---|---|
| 1. | Women ≥18 years of age |
| 2. | Stage I-III, HER2+ breast cancer at study entry, scheduled to undergo chemotherapy with one |
| | of the following regimens: (a) 5-fluorouracil, epirubicin, cyclophosphamide, followed by |
| | docetaxel and trastuzumab, (b) adriamycin, cyclophosphamide, followed by docetaxel and |
| | trastuzumab, (c) adriamycin-cyclophosphamide with weekly paclitaxel and trastuzumab, or (d) |
| | dose dense adriamycin and cyclophosphamide followed by dose dense paclitaxel and |
| | trastuzumab. One patient was enrolled as a stage III and later reclassified as stage IV, this |
| | patient was allowed to continue in the trial. |
| 3. | Ability to tolerate five 60-minute cardiac magnetic resonance examinations over 15 months |
| 4. | Ability to understand and willingness to sign a written informed consent document |

Exclusion Criteria

| | |
|---|---|
| 1. | Oncologic (or other) life expectancy <12 months |
| 2. | Participation in clinical trial of an investigational cancer drug |
| 3. | Having received previous anthracycline |
| 4. | History of myocardial infarction or previous heart failure |
| 5. | Current unstable angina, persistent atrial fibrillation or other irregular rhythm, or a history of |
| | more than mild regurgitant or stenotic valvular heart disease |
| 6. | Severely reduced renal function (GFR ≤ 30 mL/min) |
| 7. | General MRI contraindications |
| 8. | Baseline LVEF <55% by echo |
| 9. | Echocardiography image quality inadequate for strain analysis |

Participant Categorization: Adjudication of CTRCD was defined according to the CREC criteria which outlines CMR imaging measuring: (i) a ≥5% absolute reduction in LVEF from baseline to an LVEF <55% with signs or symptoms of HF, (ii) a ≥10% absolute reduction in LVEF from baseline to <55% without accompanying signs or symptoms of HF at the time points when CMR is obtained, or (iii) a fall in LVEF by >10% in patients with baseline LVEF <55%⁴⁵.

Clinical Demographics and Laboratory Results: Comprehensive clinical history was obtained at the baseline visit. Clinical laboratory parameters, including lipid profiles, hemoglobin A1c, hematocrit, cardiac troponin I (cTnI (Biomatic, EKU09460; Abbott Alinity I Series, chemiluminescent microparticle immunoassays), and b-type natriuretic peptide (BNP) (Abbott Alinity I Series, chemiluminescent microparticle immunoassays) were collected as reported by the center, with standard international reference ranges applied to decide the cut-off point for abnormal levels. Cardiac injury was defined as plasma levels of cTnI greater than the 99^{th} percentile of normal values, as per clinical guidelines.

Processing of Participant Bloodwork: At the time of cardiac imaging, peripheral blood samples (10 mL) were drawn from the cubital vein into BD Vacutainer® Blood Collection Tubes (BD Bioscience, Franklin Lakes, NJ) containing K₂EDTA and processed within three hours. The first tube was utilized for standard of care bloodwork. At no time was the plasma subjected to temperatures below 4° C. or above 25° C. Plasma was separated from whole blood through centrifugation (1,500×g, 24° C., 15 minutes) and stored at −80° C. until downstream processing. Samples were thawed on ice and subjected to sequential centrifugation of (2,500×g, 4° C., 25 minutes) to assist in the reduction of platelet counts and large particulate according to the recommended International Society on Thrombosis and Haemostasis protocol⁴⁷. Hemolysis was examined prior to downstream analysis by measuring the absorbance at 414 nm using a DS-11⁺ Spectrophotometer (DeNovix, Wilmington, Delaware, United States), with two standard deviations from the sample mean used as the threshold for sample rejection. Plasma collection protocols, processing, and quality control analyses were standardized across processing batches.

Cytokine and Chemokine Profiling: The Luminex 30-Plex Magnetic Bead Panel—Immunology Cytokine/Chemokine Assay (EMDMillipore, Burlington, USA) was employed with a Luminex 100/200 System to quantify cytokine levels in plasma samples. Frozen plasma samples (200 μL-800 μL) were thawed on ice and centrifuged at 10,000×g for 10 min at 4° C. The Bio-Plex assay was conducted on samples and serial dilutions of standards in duplicate, according to the manufacturer's instructions. Values were analyzed using a 5-Parameters Logistic non-linear regression curve model. Cytokine values deemed out of range were assigned the upper or lower limit of detection for the specific cytokine. The following analytes were assessed: Eotaxin, G-CSF, GM-CSF, IFN-α2, IFN-γ, IL-1α, IL-1β, IL-2, IL-3, IL-4, IL-5, IL-6, IL-7, IL-8, IL-10, IL-12p40, IL-12p70, IL-13, IL-15, IL-17A, ILR-1A, IP-10, MCP-1, MIP-1α, MIP-1p, MPO, TNF-α and TNF-β. Several of the analytes (i.e., IL-1α, IL-3, IL-4, IL-5, IL-6, IL-7, IL-12p40, IL-12p70, IL-13 and TNF-β) were below the level of detection in most samples and were therefore excluded from further analyses. In concert, myeloperoxidase (MPO) and growth differentiation factor-15 (GDF-15) were colorimetrically measured with Quantikine ELISAs (Catalog numbers: DMYE00B (50 μL plasma) and DGD150 (10 μL plasma), respectively, R&D Systems Inc., Minneapolis, USA).

Endothelial Protein Biomarker Analysis: Circulating levels of Angiopoietin-2 (ANGPT2; Lower limit of quantification [LLOQ]9.91 pg/mL), soluble CD62 antigen-like family member E (sE-Selectin; LLOQ 4.22 pg/mL), Endothelin-1 (ET-1; LLOQ 0.250 pg/mL), and soluble CD105/Endoglin-1 (LLOQ 21.8 pg/mL) were quantified in 50 μL of platelet free plasma samples using the Simple Plex Ella (ProteinSimple, San Jose, CA, USA) multiplex platform according to the manufacturer's instructions; all Simple Plex values are reported as the average of triplicate readings.

HTG EdgeSeq MicroRNA (miRNA) Whole Transcriptome Assay (WTA) from Plasma: Lysis of baseline plasma aliquots was facilitated by combining 30 μL plasma with equivalent (v/v) amounts of HTG Plasma Lysis Buffer (HTG Molecular, Tucson, AZ, USA) as well as 1/10^{th} (v/v) amounts of Proteinase K (HTG Molecular, Tucson, AZ, USA). The mixture was subsequently incubated for three hours at 50° C. shaking at 1,400 rpm. From each prepared sample, 35 μL were added per well to a 96-well sample plate. Human fetal brain RNA was added to one well at 25 ng/well to serve as an internal control. Samples were run on an HTG EdgeSeq Processor using the HTG EdgeSeq miRNA Whole Transcriptome Assay (HTG Molecular, Tucson, AZ, USA) to facilitate nuclease protection, whereby a pre-selected miRNA population is protected with proprietary protection probes, followed by degradation of all non-hybridized probes and non-targeted RNA by S1 nuclease. Following processing, samples were individually barcoded (using a 16-cycle PCR reaction), individually purified using AMPure XP beads (Beckman Coulter, Brea, CA, USA), and quantified using a KAPA Library Quantification kit (KAPA Biosystem, Wilmington, MA, USA). The library was sequenced on a MiSeq (Illumina, Inc., San Diego, CA) using a V3 150-cycle kit with two index reads. PhiX (Roche, Mississauga, ON, CAN) was spiked into the library at 5%; this spike-in control is standard for Illumina sequencing libraries. Data were returned from the sequencer in the form of demultiplexed FASTQ files, with one file per original well of the assay. The HTG EdgeSeq Parser (v. 5.0.535.3181, HTG Molecular, Tucson, AZ, USA) was used to align the FASTQ files to the probe list to collate the data. Data were provided as data tables of raw, quality control (QC) raw, counts per million, and median normalized.

HTG EdgeSeq MiRNA Analysis: Samples were initially analyzed using three QC metrics: (i) QC0, examining degradation of the sample; cut-off of >14% read degradation per sample as failure, (ii) QC1, insufficient read depth; read depth ≤500 k as failure, (iii) QC2, minimal expression variability; relative standard deviation of reads s 0.08 as failure. Four samples did not pass QC (3 No CTRCD, 1 CTRCD). Normalization of miRNA expression data on the remaining samples that passed QC was performed using DeSeq2⁴⁸ (v. 1.14.1) in the HTG reveal software (v.3.0.0, HTG Molecular, Tucson, AZ, USA). MiRNAs were considered detectable if they had expression levels of >5 counts per million in more than half of the samples.

Risk Assessment Using Machine Learning: Within the EMBRACE-MRI cohort leave-one-out-cross validation was performed to build the model and assess performance. Categorical features were hot encoded, with missing variables recorded as additional categorical variables, having −1.0 for numerical features. For each experiment, a random decision Forest model was fit to the training dataset and evaluated on the independent left out sample. Model performance was assessed by the area under the receiver operating characteristic (AUROC) calculated over the entire testing set. Average AUROC and 95% confidence intervals were calculated using percentile method with bootstrapping. Feature importance was estimated using SHapley Additive exPlanations (SHAP) values. Available clinical, cardiac imaging and biomarker (protein and miRNA) data that was measured across all patients at baseline were included in the model (see Supplemental Table III for a list of the features included and their definitions). MiRNA data was normalized using the median-ratio method⁴⁹. To reduce the dimensionality of the miRNA data the mean expression of the entire dataset was calculated and the top 10% of expressed miRNAs selected on for further analysis (reducing the total number to 208; Supplemental Table Ill). Data missingness is indicated in Supplemental Table IV.

SUPPLEMENTAL TABLE II
Treatment lengths for ‘CTRCD’ and ‘no CTRCD’ groups

| | No CTRCD | CTRCD |
|---|---|---|
| | (N = 76) | (N = 36) |
| Treatment-days | | |
| Anthracycline | | |
| Median (interquartile range) | 340 (336-347) | 343 (336-360) |
| Mean | 345 | 351 |
| Trastuzumab | | |
| Median (interquartile range) | 42 (42-42) | 42 (42-44) |
| Mean | 43 | 44 |

* The summary statistics are based off on the full population indicated in the column heading. There were no significant differences (p < 0.05) between no CTRCD and CTRCD groups based on t-test for continuous variables.

SUPPLEMENTAL TABLE III
Data Dictionary of Variables included in Machine Learning Analysis

| | Definition | Type of Variable |
|---|---|---|
| Clinical Variables: | | |
| age_echo | Age at time of echo | Continuous |
| bsa | Body surface area (m²) | Continuous |
| smoking | Non-smoker, smoker or ex- | Categorical |
| | smoker | |
| dm | Diabetes Melitus | Binary |
| htn | Hypertension | Binary |
| hlp | Hyperlipidemia (Cholesterol) | Binary |
| cad | Coronary Artery Disease | Binary |
| chf | Congestive Heart Failure | Binary |
| heart_failure_symptoms | Heart Failure Symptoms | Binary |
| cancer_stage | Breast Cancer Stage | Continuous (1, 2, 3, 4) |
| laterality | Laterality of Cancer | Categorial (left, right, bilateral) |
| right_stage | Cancer Stage on Right Side | Continuous |
| left_stage | Cancer Stage on Left Side | Continuous |
| hct | Hematocrit (%) | Continuous |
| tni | Troponin I (ng/mL) | Continuous |
| bnp | B-type natriuretic protein | Continuous |
| cli_sbp | Clinical Systolic Blood Pressure | Continuous |
| | (mmHg) | |
| cli_dbp | Clinical Diastolic Blood | Continuous |
| | Pressure (mmHg) | |
| cli_hr | Clinical Heart Rate (bpm) | Continuous |
| bb | Beta Blocker (yes/no) | Binary |
| acei | Angiotensin Converting | Binary |
| | Enzyme inhibitors (yes/no) | |
| arb | Angiotensin Receptor blocker | Binary |
| | (yes/no) | |
| statin | Statin (yes/no) | Binary |
| Imaging Variables: | | |
| mri_lvedv | MRI LV end diastolic volume | Continuous |
| | (ml) | |
| mri_lvesv | MRI LV end systolic volume | Continuous |
| | (ml) | |
| mri_rvedv | MRI RV end diastolic volume | Continuous |
| | (ml) | |
| mri_rvesv | MRI RV end systolic volume | Continuous |
| | (ml) | |
| mri_lvsv | MRI LV stroke volume (ml) | Continuous |
| mri_rvsv | MRI RV stroke volume (ml) | Continuous |
| mri_lvef | MRI LV ejection fraction (%) | Continuous |
| mri_rvef | MRI RV ejection fraction (%) | Continuous |
| lv_global_gcs | feature tracking-global | Continuous |
| | circumferential strain (%) | |
| lv_global_gls | feature tracking-global | Continuous |
| | logitudinal strain (%) | |
| gcsmyo | Tagging-global circumferential | Continuous |
| | strain (%) | |
| avglsmyo | Tagging-global longitudinal | Continuous |
| | strain (%) | |
| Protein Biomarkers: | | |
| e_sel | E-Selectin (pg/mL) | Continuous |
| ang_2 | Angiopoietin-2 (pg/mL) | Continuous |
| endoglin | Endoglin (pg/mL) | Continuous |
| et_1 | Endothelin-1 (pg/mL) | Continuous |
| gdf_15 | Growth/Differentiation Factor | Continuous |
| | 15 (pg/mL) | |
| mpo | Myeloperoxidase (pg/mL) | Continuous |
| MicroRNA Variables: | | |
| miRNA sequencing | Sequencing values for miRNAs | Continuous |
| | that met cut-off | |
| let_7a_5p | let_7a_5p | Continuous |
| let_7b_5p | let_7b_5p | Continuous |
| let_7c_5p | let_7c_5p | Continuous |
| let_7d_5p | let_7d_5p | Continuous |
| let_7f_5p | let_7f_5p | Continuous |
| let_7g_5p | let_7g_5p | Continuous |
| let_7i_5p | let_7i_5p | Continuous |
| miR_101_3p | miR_101_3p | Continuous |
| miR_103a_3p | miR_103a_3p | Continuous |
| miR_106b_5p | miR_106b_5p | Continuous |
| miR_107 | miR_107 | Continuous |
| miR_1207_5p | miR_1207_5p | Continuous |
| miR_1225_3p | miR_1225_3p | Continuous |
| miR_1225_5p | miR_1225_5p | Continuous |
| miR_1233_3p | miR_1233_3p | Continuous |
| miR_1234_3p | miR_1234_3p | Continuous |
| miR_1237_5p | miR_1237_5p | Continuous |
| miR_1247_3p | miR_1247_3p | Continuous |
| miR_1254 | miR_1254 | Continuous |
| miR_1255b_2_3p | miR_1255b_2_3p | Continuous |
| miR_126_3p | miR_126_3p | Continuous |
| miR_126_5p | miR_126_5p | Continuous |
| miR_1273d | miR_1273d | Continuous |
| miR_1273e | miR_1273e | Continuous |
| miR_1273h_5p | miR_1273h_5p | Continuous |
| miR_1275 | miR_1275 | Continuous |
| miR_1285_5p | miR_1285_5p | Continuous |
| miR_1287_5p | miR_1287_5p | Continuous |
| miR_1290 | miR_1290 | Continuous |
| miR_1307_3p | miR_1307_3p | Continuous |
| miR_1307_5p | miR_1307_5p | Continuous |
| miR_130a_3p | miR_130a_3p | Continuous |
| miR_142_5p | miR_142_5p | Continuous |
| miR_144_3p | miR_144_3p | Continuous |
| miR_146a_5p | miR_146a_5p | Continuous |
| miR_149_3p | miR_149_3p | Continuous |
| miR_150_5p | miR_150_5p | Continuous |
| miR_15a_5p | miR_15a_5p | Continuous |
| miR_15b_5p | miR_15b_5p | Continuous |
| miR_16_5p | miR_16_5p | Continuous |
| miR_17_5p | miR_17_5p | Continuous |
| miR_181c_5p | miR_181c_5p | Continuous |
| miR_185_5p | miR_185_5p | Continuous |
| miR_186_5p | miR_186_5p | Continuous |
| miR_187_3p | miR_187_3p | Continuous |
| miR_187_5p | miR_187_5p | Continuous |
| miR_1908_5p | miR_1908_5p | Continuous |
| miR_1913 | miR_1913 | Continuous |
| miR_1915_3p | miR_1915_3p | Continuous |
| miR_191_5p | miR_191_5p | Continuous |
| miR_193a_3p | miR_193a_3p | Continuous |
| miR_197_5p | miR_197_5p | Continuous |
| miR_199a_3p | miR_199a_3p | Continuous |
| miR_19a_3p | miR_19a_3p | Continuous |
| miR_19b_3p | miR_19b_3p | Continuous |
| miR_204_3p | miR_204_3p | Continuous |
| miR_20a_5p | miR_20a_5p | Continuous |
| miR_210_3p | miR_210_3p | Continuous |
| miR_210_5p | miR_210_5p | Continuous |
| miR_2115_3p | miR_2115_3p | Continuous |
| miR_2115_5p | miR_2115_5p | Continuous |
| miR_212_3p | miR_212_3p | Continuous |
| miR_21_5p | miR_21_5p | Continuous |
| miR_221_3p | miR_221_3p | Continuous |
| miR_222_3p | miR_222_3p | Continuous |
| miR_223_3p | miR_223_3p | Continuous |
| miR_22_3p | miR_22_3p | Continuous |
| miR_23a_3p | miR_23a_3p | Continuous |
| miR_23b_3p | miR_23b_3p | Continuous |
| miR_24_3p | miR_24_3p | Continuous |
| miR_25_3p | miR_25_3p | Continuous |
| miR_26a_5p | miR_26a_5p | Continuous |
| miR_26b_5p | miR_26b_5p | Continuous |
| miR_27a_3p | miR_27a_3p | Continuous |
| miR_27b_3p | miR_27b_3p | Continuous |
| miR_2861 | miR_2861 | Continuous |
| miR_29a_3p | miR_29a_3p | Continuous |
| miR_29b_3p | miR_29b_3p | Continuous |
| miR_29c_3p | miR_29c_3p | Continuous |
| miR_30a_5p | miR_30a_5p | Continuous |
| miR_30b_5p | miR_30b_5p | Continuous |
| miR_30c_5p | miR_30c_5p | Continuous |
| miR_30d_5p | miR_30d_5p | Continuous |
| miR_30e_5p | miR_30e_5p | Continuous |
| miR_3140_3p | miR_3140_3p | Continuous |
| miR_3140_5p | miR_3140_5p | Continuous |
| miR_3141 | miR_3141 | Continuous |
| miR_3157_5p | miR_3157_5p | Continuous |
| miR_3162_5p | miR_3162_5p | Continuous |
| miR_3180 | miR_3180 | Continuous |
| miR_3180_3p | miR_3180_3p | Continuous |
| miR_3197 | miR_3197 | Continuous |
| miR_320a | miR_320a | Continuous |
| miR_320b | miR_320b | Continuous |
| miR_320c | miR_320c | Continuous |
| miR_320d | miR_320d | Continuous |
| miR_320e | miR_320e | Continuous |
| miR_326 | miR_326 | Continuous |
| miR_339_3p | miR_339_3p | Continuous |
| miR_33b_5p | miR_33b_5p | Continuous |
| miR_345_5p | miR_345_5p | Continuous |
| miR_34b_3p | miR_34b_3p | Continuous |
| miR_34c_5p | miR_34c_5p | Continuous |
| miR_3648 | miR_3648 | Continuous |
| miR_374b_3p | miR_374b_3p | Continuous |
| miR_374c_5p | miR_374c_5p | Continuous |
| miR_3912_3p | miR_3912_3p | Continuous |
| miR_3912_5p | miR_3912_5p | Continuous |
| miR_3937 | miR_3937 | Continuous |
| miR_3940_5p | miR_3940_5p | Continuous |
| miR_3943 | miR_3943 | Continuous |
| miR_425_5p | miR_425_5p | Continuous |
| miR_4279 | miR_4279 | Continuous |
| miR_4291 | miR_4291 | Continuous |
| miR_4306 | miR_4306 | Continuous |
| miR_4309 | miR_4309 | Continuous |
| miR_4417 | miR_4417 | Continuous |
| miR_4429 | miR_4429 | Continuous |
| miR_4433b_5p | miR_4433b_5p | Continuous |
| miR_4447 | miR_4447 | Continuous |
| miR_4449 | miR_4449 | Continuous |
| miR_4458 | miR_4458 | Continuous |
| miR_4459 | miR_4459 | Continuous |
| miR_4463 | miR_4463 | Continuous |
| miR_4481 | miR_4481 | Continuous |
| miR_4486 | miR_4486 | Continuous |
| miR_4505 | miR_4505 | Continuous |
| miR_451a | miR_451a | Continuous |
| miR_4534 | miR_4534 | Continuous |
| miR_4632_5p | miR_4632_5p | Continuous |
| miR_4655_3p | miR_4655_3p | Continuous |
| miR_4664_3p | miR_4664_3p | Continuous |
| miR_4667_5p | miR_4667_5p | Continuous |
| miR_4690_5p | miR_4690_5p | Continuous |
| miR_4706 | miR_4706 | Continuous |
| miR_4713_3p | miR_4713_3p | Continuous |
| miR_4745_3p | miR_4745_3p | Continuous |
| miR_4763_3p | miR_4763_3p | Continuous |
| miR_4784 | miR_4784 | Continuous |
| miR_4793_5p | miR_4793_5p | Continuous |
| miR_4795_3p | miR_4795_3p | Continuous |
| miR_4795_5p | miR_4795_5p | Continuous |
| miR_4800_3p | miR_4800_3p | Continuous |
| miR_484 | miR_484 | Continuous |
| miR_486_5p | miR_486_5p | Continuous |
| miR_5001_5p | miR_5001_5p | Continuous |
| miR_5196_5p | miR_5196_5p | Continuous |
| miR_541_3p | miR_541_3p | Continuous |
| miR_5585_3p | miR_5585_3p | Continuous |
| miR_5587_3p | miR_5587_3p | Continuous |
| miR_561_3p | miR_561_3p | Continuous |
| miR_561_5p | miR_561_5p | Continuous |
| miR_5694 | miR_5694 | Continuous |
| miR_5739 | miR_5739 | Continuous |
| miR_574_3p | miR_574_3p | Continuous |
| miR_574_5p | miR_574_5p | Continuous |
| miR_6085 | miR_6085 | Continuous |
| miR_6088 | miR_6088 | Continuous |
| miR_6124 | miR_6124 | Continuous |
| miR_6126 | miR_6126 | Continuous |
| miR_6131 | miR_6131 | Continuous |
| miR_6165 | miR_6165 | Continuous |
| miR_654_5p | miR_654_5p | Continuous |
| miR_658 | miR_658 | Continuous |
| miR_671_5p | miR_671_5p | Continuous |
| miR_6716_3p | miR_6716_3p | Continuous |
| miR_6727_5p | miR_6727_5p | Continuous |
| miR_6741_5p | miR_6741_5p | Continuous |
| miR_6750_5p | miR_6750_5p | Continuous |
| miR_6756_5p | miR_6756_5p | Continuous |
| miR_6765_5p | miR_6765_5p | Continuous |
| miR_6769b_3p | miR_6769b_3p | Continuous |
| miR_6775_5p | miR_6775_5p | Continuous |
| miR_6778_5p | miR_6778_5p | Continuous |
| miR_6780b_5p | miR_6780b_5p | Continuous |
| miR_6781_5p | miR_6781_5p | Continuous |
| miR_6789_5p | miR_6789_5p | Continuous |
| miR_6794_5p | miR_6794_5p | Continuous |
| miR_6796_3p | miR_6796_3p | Continuous |
| miR_6798_5p | miR_6798_5p | Continuous |
| miR_6799_5p | miR_6799_5p | Continuous |
| miR_6800_5p | miR_6800_5p | Continuous |
| miR_6802_5p | miR_6802_5p | Continuous |
| miR_6803_5p | miR_6803_5p | Continuous |
| miR_6810_3p | miR_6810_3p | Continuous |
| miR_6819_3p | miR_6819_3p | Continuous |
| miR_6821_5p | miR_6821_5p | Continuous |
| miR_6825_3p | miR_6825_3p | Continuous |
| miR_6845_5p | miR_6845_5p | Continuous |
| miR_6852_3p | miR_6852_3p | Continuous |
| miR_6869_5p | miR_6869_5p | Continuous |
| miR_6870_3p | miR_6870_3p | Continuous |
| miR_6870_5p | miR_6870_5p | Continuous |
| miR_6873_3p | miR_6873_3p | Continuous |
| miR_6875_5p | miR_6875_5p | Continuous |
| miR_6892_3p | miR_6892_3p | Continuous |
| miR_7107_5p | miR_7107_5p | Continuous |
| miR_7108_5p | miR_7108_5p | Continuous |
| miR_7111_5p | miR_7111_5p | Continuous |
| miR_7114_3p | miR_7114_3p | Continuous |
| miR_7150 | miR_7150 | Continuous |
| miR_764 | miR_764 | Continuous |
| miR_7845_5p | miR_7845_5p | Continuous |
| miR_8069 | miR_8069 | Continuous |
| miR_874_3p | miR_874_3p | Continuous |
| miR_92a_3p | miR_92a_3p | Continuous |
| miR_92b_3p | miR_92b_3p | Continuous |
| miR_93_5p | miR_93_5p | Continuous |

SUPPLEMENTAL TABLE IV
Summary of Missing Data Points for Variables
used in MachineLearning Model

| | Missing |
|---|---|
| | Data Points |
| Clinical Variables: | |
| age_echo | 0 |
| bsa | 0 |
| smoking | 0 |
| dm | 0 |
| htn | 0 |
| hlp | 0 |
| cad | 0 |
| chf | 0 |
| heart_failure_symptoms | 0 |
| cancer_stage | 0 |
| laterality | 0 |
| right_stage | 78 |
| left_stage | 56 |
| hct | 0 |
| tni | 0 |
| bnp | 2 |
| cli_sbp | 0 |
| cli_dbp | 0 |
| cli_hr | 0 |
| bb | 0 |
| acei | 0 |
| arb | 0 |
| statin | 0 |
| Imaging Variables: | |
| mri_lvedv | 0 |
| mri_lvesv | 0 |
| mri_rvedv | 1 |
| mri_rvesv | 1 |
| mri_lvsv | 0 |
| mri_rvsv | 1 |
| mri_lvef | 0 |
| mri_rvef | 1 |
| lv_global_gcs | 1 |
| lv_global_gls | 0 |
| gcsmyo | 14 |
| avglsmyo | 17 |
| Protein Biomarkers: | |
| e_sel | 2 |
| ang_2 | 0 |
| endoglin | 0 |
| et_1 | 0 |
| gdf_15 | 6 |
| mpo | 7 |
| MicroRNA Variables: | |
| miRNA sequencing | 4 |

Blinding Procedures: Blinding was performed at the stage of assay initiation where possible (i.e. loading of samples and sequencing).

Data Visualization and Statistical Analysis: Descriptive Analysis—Clinical characteristics were analyzed using summary statistics. Continuous variables were described using median and interquartile range (IQR), and dichotomous or polytomous variables were described using frequencies. Between-group differences were evaluated using Wilcoxon rank-sum tests for continuous variables and Fisher's exact tests for dichotomous/polytomous variables. Biomarker Analysis—The normality of the distributions was evaluated using the D'Agostino-Pearson test. If distributions were normal, unpaired Student's t-tests was used for two-group comparisons and two-way analysis of variance analysis (ANOVA) with Tukey's post-hoc test was used when multiple groups across several time-points were being compared. If distributions were nonnormal, the Mann-Whitney U-test was used for the analysis of two groups and the Kruskal-Wallis test with Dunn's post hoc test for multiple-group comparisons. P values of <0.05 were considered statistically significant and indicated in the graphs as reported by the analysis software with significance thresholds of P<0.05, P<0.01, P<0.001, and P<0.0001 denoted as *, **, ***, **** respectively. Power calculations were not performed to determine sample size and group sizes as they were determined based on similar publications in the field. MiRNA pathway analysis was conducted using BioCarta/KEGG/Reactome databases (miRanDa) and tested for enrichment by a hypergeometric test with adjustment for multiple comparisons using the Benjamini-Hochberg False Discovery Rate (FDR), with P<0.05 considered to be statistically enriched in a gene set of interest⁵⁰⁻⁵². Although many hypotheses were tested throughout the manuscript, no experiment-wide multiple test correction was applied. Unless indicated otherwise, graphs depict averaged values of independent data points based on technical replicates and have error bars displayed as mean+/−standard deviation (±S.D.). Data were analyzed with GraphPad Prism 9.0.0 for MacOS (GraphPad Software, Inc., La Jolla, CA, USA; Biomarker Multiple Comparisons). Final figures were assembled for publication purposes using Adobe Illustrator (v27).

### Example 2: Results of Example 1

Patient Demographics and Identification of Patients with CTRCD—The study included 136 women (age: 51.0±9.2 years) undergoing therapy for HER2⁺ breast cancer (Table I). During treatment, the lowest cumulative ejection fraction was reached three months into trastuzumab therapy (i.e., TP3, FIG. 1B). Patients were retrospectively divided into ‘no CTRCD’ and ‘CTRCD’ groups based on CMR imaging during treatment (see Methods). Importantly, there were no significant differences in LVEF at baseline; however, differences between groups could be detected as early as TP2 (FIG. 1B). A total of 37 (27.2%) patients developed CTRCD.

At baseline there were no significant differences in age, blood pressure or cardiovascular laboratory parameters nor concurrent cardiovascular therapies between patients who developed CTRCD and those who did not (Table I). Additionally, both cancer staging as well as chemotherapeutic regimens were similar between the two groups (Table I) as was treatment length (Supplemental Table II). Cardiovascular (CV) risk factors were relatively minimal, with hyperlipidemia being the only CV risk factor that was different between groups, with 8 patients (5.9%) in the ‘CTRCD’ group and 6 patients (4.4%) in the ‘no CTRCD’ group having this risk factor. No patients had coronary artery disease or congestive heart failure at baseline.

Lack of Distinguishing Classic and Emerging Cardiac Damage Biomarkers in Patients that Developed CTRCD—The first three timepoints of treatment (i.e., TP1-3) were focused on to assess early biomarkers of CTRCD. Traditional circulating cardiac biomarkers (FIG. 1C) were first assessed. Cardiac troponin I (cTnI) levels were modestly higher in the ‘no CTRCD’ group compared to the ‘CTRCD’ group at baseline (2.2 ng/mL in ‘CTRCD’ vs. 3.7 ng/mL in ‘no CTRCD’ group, but levels were low overall. While levels of cTnI increased during treatment (i.e., TP2 and TP3), there were no significant differences between ‘CTRCD’ and ‘no CTRCD’ groups at these timepoints. There were also no significant differences in B-type natriuretic peptide (BNP) between groups, although levels increased during treatment (i.e., TP2 and TP3). Likewise, GDF-15, a marker of oxidative stress, inflammation and mechanical strain in the heart³¹, increased during treatment (i.e., TP2 and TP3), but there were no significant differences between groups.

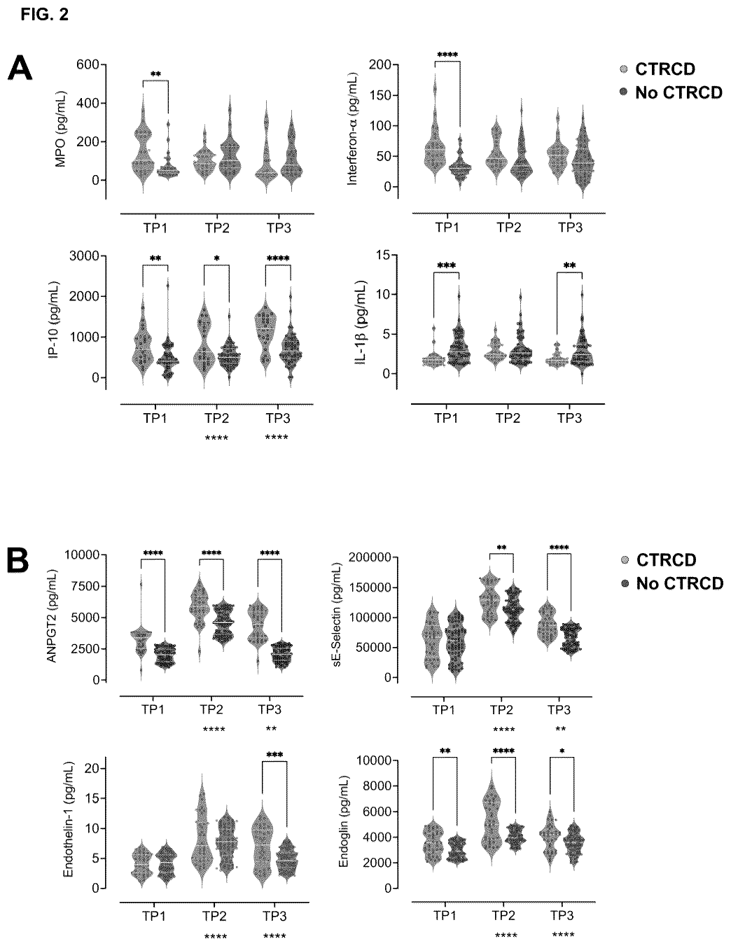

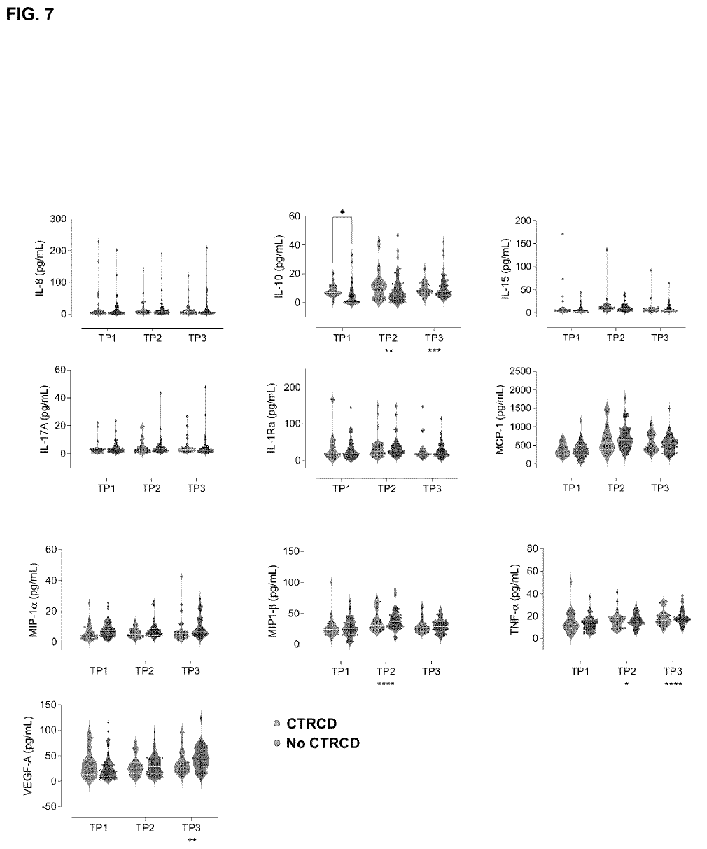

Patients That Developed CTRCD Had Elevated Circulating Levels of Inflammatory and Endothelial-Centric Dysfunction Markers Pre-Treatment and Early During Treatment—Circulating markers of immune activation as well as angiogenic factors were measured at baseline (TP1) and during the early stages of cancer treatment (i.e., TP2 and TP3) in a subset of patients (FIG. 2A, FIG. 7). Of these, G-CSF, IL-10, IP-10, MCP-1, MIP-1β and TNF-α were increased during treatment (i.e., TP2 and/or TP3). Notably, IFN-α, IL-10, IP-10 and MPO were elevated in the ‘CTRCD’ group compared to the ‘no CTRCD’ group at baseline prior to cancer treatment (FIG. 2A, FIG. 7). IP-10 was elevated in the ‘CTRCD’ group at baseline and throughout treatment (FIG. 2A).

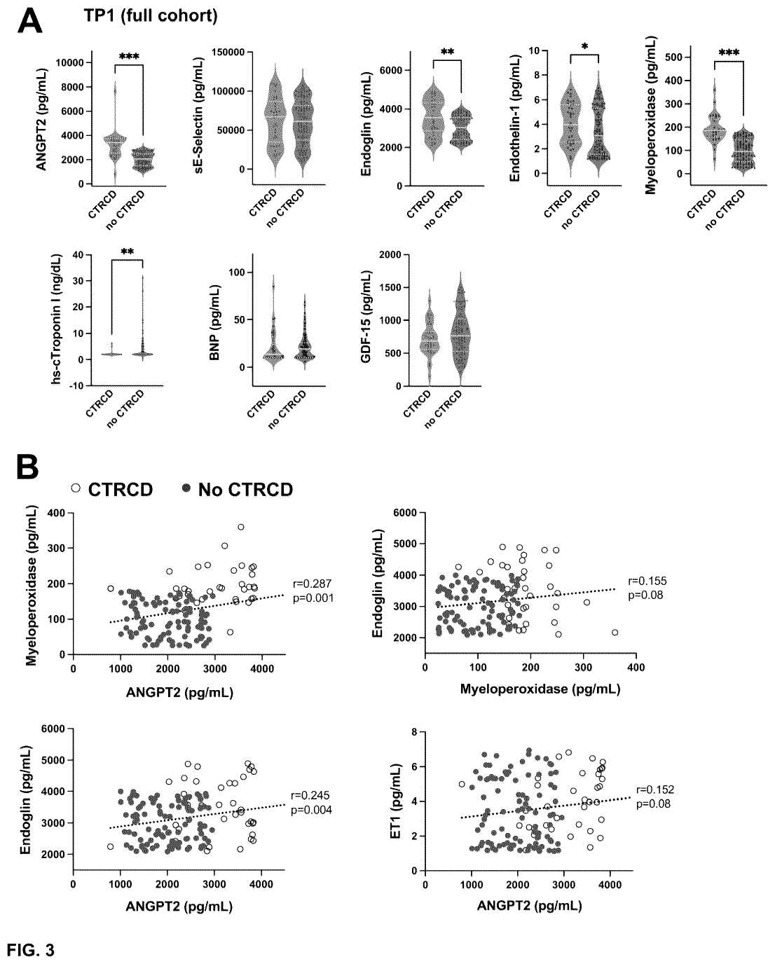

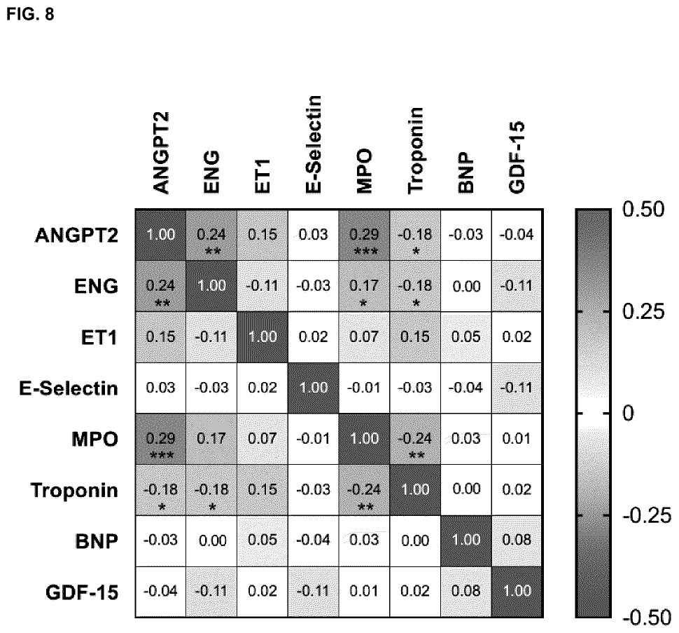

The endothelial activation markers, Angiopoietin-2 [ANGPT2], soluble E-selectin [sE-selectin], Endoglin [ENG], and Endothelin-1 [ET-1] were measured in a subset of patients. Notably, all markers were robustly increased during treatment (i.e., TP2 and TP3, FIG. 2B). Among these markers, two were significantly different at baseline between patients who developed CTRCD compared to those who did not (i.e., ANGPT2 and ENG, FIG. 2B) and these remained elevated at early treatment timepoints. In contrast, sE-Selectin and ET-1 were elevated during therapy (TP2/TP3 and TP3, respectively) in patients that developed CTRCD compared to those that did not (FIG. 2B). All endothelial-centric markers, cardiac markers, along with MPO (a marker of neutrophil activation and inflammation), were subsequently measured in all pre-treatment baseline samples, which revealed a significant elevation in the levels of ANGPT2, ENG, ET-1 and MPO in patients who developed CTRCD (FIG. 3A). Correlation plots revealed that ANGPT2 levels were correlated with ENG and MPO in baseline samples (FIG. 3B; FIG. 8).

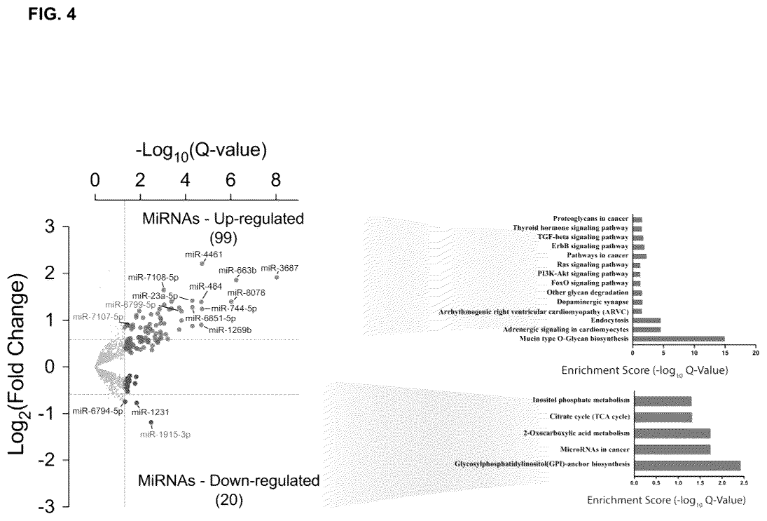

Assessment of Pre-Treatment Circulating Plasma miRNA Expression Identified Distinct Signatures in Patients that will Later Develop CTRCD—Plasma miRNAs was profiled in all pre-treatment samples to identify meaningful differences in miRNA composition. Differential expression analysis highlighted 119 differentially expressed miRNAs (n=99 up-regulated and n=20 down-regulated) between the two sub-groups at baseline (FIG. 4, Supplemental Table V), demonstrating that miRNAs can stratify patients for CTRCD risk. Pathway analysis of the predicted mRNA targets of differentially expressed miRNAs that were up-regulated in the ‘CTRCD’ group using DIANA-mirPath suggested broad over-representation of pathways related to cardiomyocyte function (i.e., ErbB2 signaling and arrhythmogenic right ventricular cardiomyopathy) as well as inflammatory pathways (i.e., TGF-beta signaling and Ras signaling, FIG. 4). Analysis of miRNAs that were down-regulated in the ‘CTRCD’ group revealed an enrichment of pathways involved in glycosylphosphatidylinositol-anchor biosynthesis and miRNAs in cancer (FIG. 4).

SUPPLEMENTAL TABLE V
Differentially Abundant Plasma MiRNAs in Baseline Samples

| | Mean | Mean | | |
|---|---|---|---|---|
| | Normalized | Normalized | | |
| | Counts | Counts ‘no | Fold- | Adjusted |
| | ‘CTRCD’ | CTRCD’ | change | p-value |
| miR-4461 | 4487 | 2916 | 4.61 | 1.87E−05 |
| miR-3687 | 2870 | 728 | 3.75 | 9.31E−09 |
| miR-663b | 3331 | 924 | 3.61 | 5.76E−07 |
| miR-7108-5p | 10816 | 3472 | 3.12 | 9.29E−04 |
| miR-23a-5p | 737 | 277 | 2.67 | 5.02E−05 |
| miR-8078 | 980 | 373 | 2.63 | 9.45E−07 |
| miR-6723-5p | 770 | 618 | 2.63 | 4.18E−04 |
| miR-484 | 4933 | 2626 | 2.62 | 1.98E−05 |
| miR-3648 | 10240 | 4073 | 2.51 | 8.74E−04 |
| miR-6851-5p | 876 | 363 | 2.42 | 5.02E−05 |
| miR-652-3p | 2350 | 1502 | 2.41 | 1.91E−04 |
| miR-27a-5p | 554 | 235 | 2.37 | 4.18E−04 |
| miR-744-5p | 356 | 25 | 2.36 | 1.87E−05 |
| miR-221-3p | 6305 | 4598 | 2.35 | 1.40E−03 |
| miR-6812-5p | 2444 | 1045 | 2.34 | 5.22E−04 |
| miR-146a-5p | 9469 | 6309 | 2.30 | 1.80E−03 |
| miR-486-5p | 22780 | 9946 | 2.29 | 1.10E−02 |
| miR-6799-5p | 15009 | 8162 | 2.28 | 1.51E−04 |
| miR-1273h-5p | 22620 | 10277 | 2.20 | 1.70E−03 |
| miR-6869-5p | 3950 | 1816 | 2.18 | 3.30E−03 |
| miR-19b-3p | 19942 | 9293 | 2.15 | 1.52E−02 |
| miR-92a-3p | 24250 | 11632 | 2.08 | 1.52E−02 |
| miR-6741-5p | 10636 | 7233 | 2.07 | 1.30E−03 |
| miR-127-3p | 331 | 343 | 2.07 | 7.50E−03 |
| miR-920 | 707 | 351 | 2.02 | 1.20E−03 |
| miR-4539 | 958 | 482 | 1.99 | 1.51E−04 |
| miR-3674 | 814 | 429 | 1.90 | 8.74E−04 |
| miR-328-3p | 1319 | 1175 | 1.90 | 1.30E−03 |
| miR-5010-5p | 479 | 253 | 1.89 | 3.80E−03 |
| miR-25-3p | 8191 | 4357 | 1.88 | 3.90E−02 |
| miR-8069 | 5170 | 2766 | 1.87 | 2.16E−02 |
| miR-1299 | 1235 | 663 | 1.87 | 3.62E−02 |
| miR-1269b | 281 | 150 | 1.86 | 1.98E−05 |
| miR-425-3p | 818 | 722 | 1.85 | 2.00E−03 |
| miR-7107-5p | 8126 | 4423 | 1.84 | 3.25E−02 |
| miR-6846-5p | 206 | 112 | 1.83 | 5.02E−05 |
| miR-339-5p | 1558 | 1295 | 1.80 | 4.50E−03 |
| miR-6894-5p | 1051 | 584 | 1.80 | 2.08E−02 |
| miR-4459 | 14570 | 8095 | 1.80 | 4.75E−02 |
| miR-143-3p | 881 | 797 | 1.79 | 5.70E−03 |
| miR-151a-3p | 1401 | 1208 | 1.79 | 1.01E−02 |
| miR-33a-5p | 493 | 417 | 1.76 | 3.70E−03 |
| miR-6127 | 1567 | 896 | 1.75 | 2.43E−02 |
| miR-1229-5p | 206 | 118 | 1.74 | 1.91E−04 |
| miR-6829-5p | 362 | 212 | 1.71 | 3.00E−03 |
| miR-4496 | 580 | 343 | 1.69 | 5.90E−03 |
| miR-8089 | 453 | 268 | 1.68 | 1.30E−03 |
| miR-501-3p | 278 | 164 | 1.65 | 4.50E−03 |
| miR-370-3p | 327 | 264 | 1.64 | 2.00E−03 |
| miR-6848-5p | 164 | 101 | 1.62 | 7.39E−04 |
| miR-1244 | 245 | 152 | 1.61 | 1.20E−03 |
| miR-3679-5p | 195 | 125 | 1.56 | 2.30E−03 |
| miR-502-3p | 272 | 170 | 1.55 | 9.20E−03 |
| miR-8085 | 307 | 197 | 1.55 | 1.17E−02 |
| miR-4732-3p | 406 | 259 | 1.55 | 1.20E−02 |
| miR-4442 | 470 | 305 | 1.54 | 5.90E−03 |
| miR-342-5p | 134 | 87 | 1.54 | 1.01E−02 |
| miR-1182 | 148 | 95 | 1.53 | 1.20E−03 |
| miR-1291 | 529 | 346 | 1.53 | 2.74E−02 |
| miR-4687-3p | 264 | 174 | 1.51 | 6.02E−04 |
| miR-532-3p | 309 | 199 | 1.51 | 1.40E−03 |
| miR-4507 | 768 | 507 | 1.51 | 3.38E−02 |
| miR-6716-5p | 319 | 212 | 1.50 | 9.20E−03 |
| miR-939-5p | 248 | 166 | 1.49 | 4.50E−03 |
| miR-532-5p | 354 | 234 | 1.49 | 2.87E−02 |
| miR-6757-5p | 213 | 143 | 1.48 | 2.10E−03 |
| miR-6779-5p | 231 | 156 | 1.48 | 7.00E−03 |
| miR-4419b | 338 | 229 | 1.47 | 3.83E−02 |
| miR-664b-3p | 138 | 94 | 1.46 | 4.50E−03 |
| miR-6865-5p | 240 | 164 | 1.46 | 3.26E−02 |
| miR-6780a-5p | 192 | 131 | 1.45 | 1.70E−03 |
| miR-4646-5p | 467 | 322 | 1.45 | 8.50E−03 |
| miR-1301-3p | 138 | 114 | 1.45 | 1.03E−02 |
| miR-660-5p | 318 | 215 | 1.45 | 2.74E−02 |
| miR-1236-5p | 174 | 119 | 1.44 | 4.50E−03 |
| miR-6747-5p | 193 | 134 | 1.44 | 4.60E−03 |
| miR-3064-5p | 134 | 92 | 1.43 | 1.70E−03 |
| miR-296-3p | 266 | 186 | 1.43 | 3.48E−02 |
| miR-7106-5p | 437 | 303 | 1.43 | 3.90E−02 |
| miR-6776-5p | 306 | 214 | 1.42 | 3.82E−02 |
| miR-3689a-3p | 267 | 111 | 1.40 | 2.81E−02 |
| miR-2278 | 135 | 96 | 1.39 | 2.11E−02 |
| miR-6862-5p | 188 | 135 | 1.37 | 2.90E−03 |
| miR-181b-5p | 352 | 256 | 1.37 | 4.23E−02 |
| miR-3689b-3p | 126 | 92 | 1.35 | 2.01E−02 |
| miR-1183 | 122 | 90 | 1.35 | 2.74E−02 |
| miR-4253 | 372 | 276 | 1.34 | 4.90E−02 |
| miR-4649-5p | 134 | 101 | 1.33 | 7.00E−03 |
| miR-3147 | 189 | 142 | 1.32 | 2.81E−02 |
| miR-3131 | 160 | 120 | 1.32 | 3.38E−02 |
| miR-6500-3p | 201 | 114 | 1.32 | 4.24E−02 |
| miR-34a-5p | 218 | 162 | 1.31 | 1.41E−02 |
| miR-1911-3p | 134 | 101 | 1.31 | 1.44E−02 |
| miR-421 | 142 | 108 | 1.31 | 3.39E−02 |
| miR-6882-3p | 170 | 129 | 1.29 | 1.01E−02 |
| miR-6772-5p | 179 | 137 | 1.29 | 4.26E−02 |
| miR-6735-5p | 170 | 130 | 1.28 | 3.10E−03 |
| miR-1271-5p | 136 | 108 | 1.25 | 2.87E−02 |
| miR-4268 | 151 | 120 | 1.23 | 2.53E−02 |
| miR-548aw | 173 | 202 | −1.14 | 2.90E−02 |
| miR-129-2-3p | 122 | 144 | −1.16 | 1.52E−02 |
| miR-145-3p | 210 | 257 | −1.19 | 3.83E−02 |
| miR-556-3p | 265 | 324 | −1.21 | 3.83E−02 |
| miR-6800-3p | 276 | 340 | −1.22 | 3.12E−02 |
| miR-6884-3p | 281 | 351 | −1.24 | 3.09E−02 |
| miR-15b-3p | 271 | 346 | −1.26 | 3.62E−02 |
| miR-206 | 225 | 290 | −1.27 | 4.75E−02 |
| miR-181a-3p | 250 | 321 | −1.27 | 4.99E−02 |
| miR-6075 | 146 | 189 | −1.28 | 1.68E−02 |
| miR-18b-3p | 242 | 317 | −1.30 | 3.62E−02 |
| miR-153-3p | 262 | 351 | −1.32 | 3.82E−02 |
| miR-141-5p | 401 | 545 | −1.35 | 3.62E−02 |
| miR-4783-3p | 185 | 248 | −1.35 | 4.36E−02 |
| miR-1306-5p | 497 | 671 | −1.35 | 4.79E−02 |
| miR-6810-3p | 1024 | 1404 | −1.37 | 4.79E−02 |
| miR-877-3p | 799 | 1153 | −1.44 | 3.62E−02 |
| miR-6794-5p | 21675 | 36351 | −1.68 | 4.69E−02 |
| miR-1231 | 453 | 776 | −1.71 | 1.44E−02 |
| miR-1915-3p | 10750 | 24468 | −2.28 | 3.30E−03 |

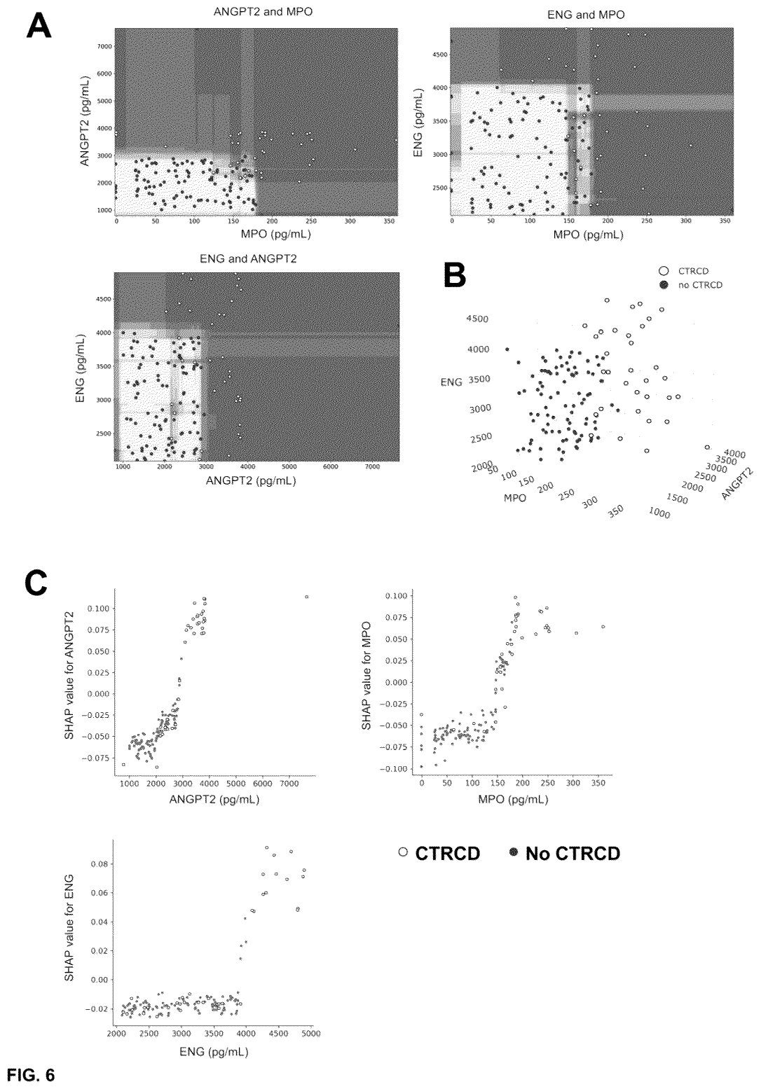

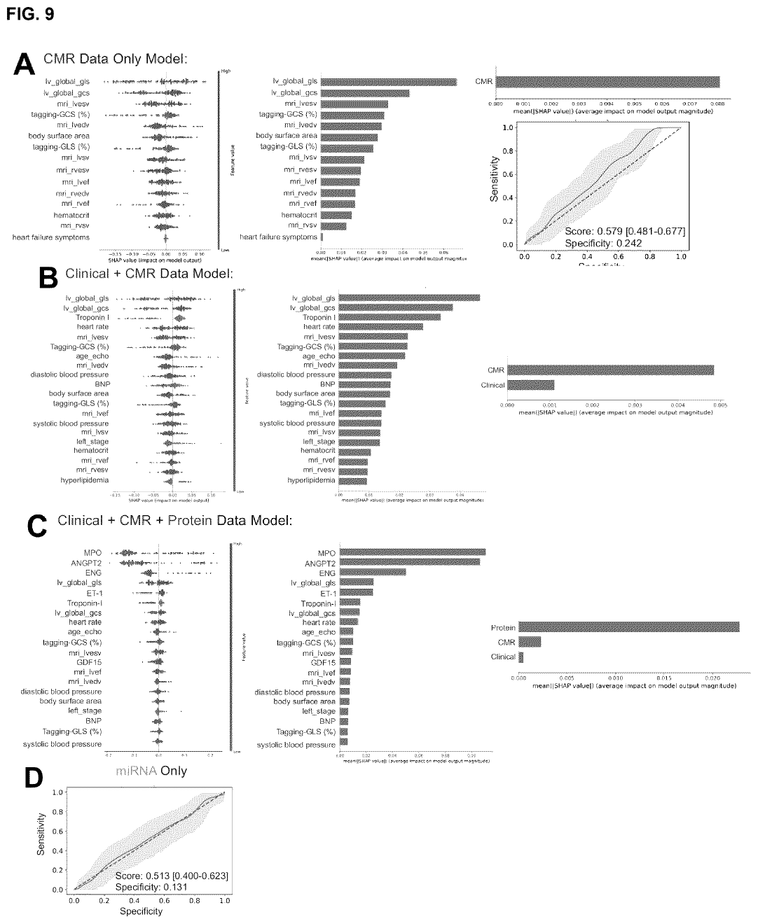

Machine Learning Highlighted that Circulating Biomarkers Increased Predictive Power Above Clinical and Cardiac Imaging Measures Alone—The high dimensionality of the pre-treatment baseline datasets that was generated was taken advantage of to develop a machine learning model to predict the risk of developing CTRCD during treatment. An unbiased approach was utilized, which combined clinical features, cardiac magnetic resonance (CMR) imaging parameters, protein and miRNA expression data that were available in all patients at baseline (Supplemental Table III). Leave-one-out cross validation was performed to train a set of Random Forest models, similar to what was done previously for clinical and biomarker data of COVID-19-related mortality³⁹. A low area under the receiver operating characteristic (AUROC) was observed using clinical features alone (0.666 [confidence interval=0.564-0.772]; specificity=0.121) (FIG. 5A) or for CMR features alone (0.579 [0.481-0.677]; specificity=0.242) (FIG. 9A). Addition of CMR to the clinical data led to a modest improvement in score (0.673 [0.572-0.775]; specificity=0.232) (FIG. 5A, FIG. 9B). Remarkably, addition of protein biomarkers to clinical and CMR data markedly improved the AUROC score (0.968 [0.939-0.990]; specificity=0.889) (FIG. 5A, FIG. 9C). Addition of miRNA data to clinical, CMR and protein data did not further improve the model (0.894 [0.829-0.947]; specificity=0.646) (FIG. 5A). MiRNAs on their own had low AUROC scores (FIG. 9D) and did not add to the Clinical+CMR+Protein values (FIG. 5A). In contrast, utilizing protein data alone led to a high AUROC score (0.979 [0.953-0.995]; specificity=0.939), but using only the top performing protein biomarker, MPO, had a much lower score (0.840 [0.746-0.921]; specificity=0.424) (FIG. 5A), suggesting that there is benefit to measuring multiple protein biomarkers. Assessing the contribution of categories of features to the performance of the full model revealed that protein biomarkers had the largest impact, with CMR and miRNA data also contributing (FIG. 5B). Relatively little impact was contributed by clinical data. Assessing the impact of individual features revealed that MPO, ANGPT2 and ENG were by far the highest-ranking features, as indicated by SHAP value (FIGS. 5C and D). However, CMR features such as left ventricular global longitudinal and circumferential strain, and miRNA features such as miR-6727-5p and miR-1915-3p were among the top 10 features (FIGS. 5C and D). We approximated how the model prediction of CTRCD changes as a function of pairwise combinations of protein biomarker concentrations, suggesting cut-offs of concentration that are associated with heightened risk (FIG. 6A). Plotting MPO, ANGPT2 and ENG concentration together in 3D, revealed clear separation between ‘CTRCD’ and ‘no CTRCD’ groups. Finally, plotting of the SHAP value verses concentration for ANGPT2, MPO, ENG and ET-1, highlighted concentrations of the analytes at which there was a dynamic change in model output (FIG. 6C).

### Example 3

Early diagnosis of CTRCD remains a challenge due to a lack of easily accessible diagnostic methods that are both sensitive and specific. Importantly, early intervention has been shown to have clinical benefit. The novel approach demonstrated herein has shown that inflammatory markers (e.g., MPO, IP-10, Interferon-α) and endothelial activation/dysfunction markers (e.g., ANGPT2, ENG, E-Selectin, ET-1) are significantly different between patients who develop CTRCD compared to those who do not. Importantly, a subset of these markers (ANGPT2, ENG, MPO, IP-10, Interferon-α) are already elevated in patients that later develop CTRCD, even before cancer treatment is initiated. In contrast, cardiac biomarkers (Troponin I, BNP, GDF-15) are uninformative in this cohort. Although less informative than protein biomarkers, whole transcriptomic miRNA analyses reveal that miRNA expression in plasma at baseline may also risk-stratify patients and uncovers potential pathways that may contribute to CTRCD. Because of the high dimensionality of complete baseline clinical, cardiac imaging and biomarker data that were generated in this study, a Random Forest Machine Learning Model was utilized to identify the strongest predictors of CTRCD outcome based on all available baseline data. Strikingly, levels of MPO, ANGPT2 and ENG were by far the best predictors and were further validated in a separate cohort. Taken together, this study revealed novel biomarkers and potential mechanisms of cardiac dysfunction in breast cancer patients treated with anthracyclines and trastuzumab.

Strikingly, many of the inflammatory and EC activation markers that were identified (e.g., MPO, ANGPT2, ENG, ET-1, IP-10, Interferon-α) were already elevated prior to cancer treatment, implying that they may be indicative of sub-clinical systemic vascular inflammation that predisposes towards cardiac dysfunction upon initiation of cancer therapy. As the machine learning analysis did not identify strong associations between CTRCD and baseline clinical factors, lifestyle factors, or cardiac medications, it remains unclear what is responsible for the systemic inflammatory state in patients that go on to develop CTRCD.

In addition to circulating inflammatory and EC dysfunction markers, this study also revealed a compendium of miRNAs that were altered in plasma prior to cancer therapy. Since miRNAs control gene expression through targeting mRNAs, pathways regulated by the identified miRNAs were able to be predicted, providing potential mechanistic insight. The ErbB2 pathway was particularly interesting as myocardial ErbB2 is part of an endothelium-controlled Neuregulin-1/ErbB signaling axis, whereby Neuregulin-1, secreted from cardiac microvascular endothelial cells, binds to ErbB receptors in the myocardial tissue⁶⁷.

Taken together, this study has provided the most comprehensive, high-resolution interrogation of a diverse group of cardiovascular markers between patients who developed CTRCD and those who did not. Notably, using a machine learning approach, multiple types of available baseline data were integrated, which revealed novel markers and biology of CTRCD. This study suggests that pre-existing inflammation and EC dysfunction may provide a strong underlying susceptibility to cardiac damage during cancer therapy. Understanding whether and how changes in the endothelial secretome mediate the pathogenesis of CTRCD may open new avenues in understanding the mechanistic interplay between the endothelium and cardiomyocytes, as well as reveal novel biomarkers and treatment targets for CTRCD.

### Example 4

The markers are being validated in samples from three clinical studies. The SUCCOUR and SPARE-HF are two studies including HER2+ breast cancer patients. The SUCCOUR study assessed strain surveillance during treatment (68), while SPARE-HF assessed treatment with statins (69).

The validation cohort is 116 patients the majority of which have with HER2+ breast cancer, who were treated with anthracyclines and trastuzumab. Thirty-one patients of the cohort had other cancers e.g. lymphoma. Various biomarkers identified herein including MPO, ANGPT2, ET1, ENG, E-Selectin, GDF-15, are being assessed this validation cohort.

Initial analysis of a subset of markers for 3 or 4 patients that developed CTRCD compared to patients that did not develop CTRCD, found the average level of ANGPT2, E-selectin, Endoglin, GDF-15, ET-1 and MPO to be increased in patients with CTRCD.

### REFERENCES

1. Society C C. Canadian Cancer Statistics Advisory Committee. Canadian Cancer Statistics 2018. 2018.
2. Thavendiranathan P, Abdel-Qadir H, Fischer H D, et al. Breast Cancer Therapy-Related Cardiac Dysfunction in Adult Women Treated in Routine Clinical Practice: A Population-Based Cohort Study. 2016; 34:2239-46.
3. Abdel-Qadir H, Austin P C, Lee D S, et al. A population-based study of cardiovascular mortality following early-stage breast cancer. 2017; 2:88-93.
4. Cardinale D, Colombo A, Lamantia G, et al. Anthracycline-induced cardiomyopathy: clinical relevance and response to pharmacologic therapy. 2010; 55:213-20.
5. Chen J, Long J B, Hurria A, Owusu C, Steingart R M, Gross C P. Incidence of heart failure or cardiomyopathy after adjuvant trastuzumab therapy for breast cancer. J Am Coll Cardiol 2012; 60:2504-12.
6. Telli M L, Hunt S A, Carlson R W, Guardino A E. Trastuzumab-related cardiotoxicity: calling into question the concept of reversibility. J Clin Oncol 2007; 25:3525-33.
7. Yeh E T, Bickford C L. Cardiovascular complications of cancer therapy: incidence, pathogenesis, diagnosis, and management. J Am Coll Cardiol 2009; 53:2231-47.
8. Moja L, Tagliabue L, Balduzzi S, et al. Trastuzumab containing regimens for early breast cancer. Cochrane Database Syst Rev 2012; 4:CD006243.
9. Abdel-Qadir H, Austin P C, Lee D S, et al. A Population-Based Study of Cardiovascular Mortality Following Early-Stage Breast Cancer Cardiovascular Mortality Following Early-Stage Breast Cancer Cardiovascular Mortality Following Early-Stage Breast Cancer. JAMA Cardiology 2017; 2:88-93.
10. Thavendiranathan P, Abdel-Qadir H, Fischer H D, et al. Risk-Imaging Mismatch in Cardiac Imaging Practices for Women Receiving Systemic Therapy for Early-Stage Breast Cancer: A Population-Based Cohort Study. 2018; 36:2980-7.
11. Calvillo-Argüelles O, Abdel-Qadir H, Michalowska M, et al. Cardioprotective Effect of Statins in Patients With HER2-Positive Breast Cancer Receiving Trastuzumab Therapy. 2019; 35:153-9.
12. Armenian S H, Lacchetti C, Barac A, et al. Prevention and monitoring of cardiac dysfunction in survivors of adult cancers: American Society of Clinical Oncology Clinical Practice Guideline. 2016.
13. Ewer M S, Lenihan D J. Left ventricular ejection fraction and cardiotoxicity: is our ear really to the ground? J Clin Oncol 2008; 26:1201-3.
14. Gulati G, Zhang K W, Scherrer-Crosbie M, Ky B. Cancer and cardiovascular disease: the use of novel echocardiography measures to predict subsequent cardiotoxicity in breast cancer treated with anthracyclines and trastuzumab. Curr Heart Fail Rep 2014; 11:366-73.
15. Cardinale D, Sandri M T, Martinoni A, et al. Myocardial injury revealed by plasma troponin I in breast cancer treated with high-dose chemotherapy. Ann Oncol 2002; 13:710-5.
16. Dodos F, Halbsguth T, Erdmann E, Hoppe U C. Usefulness of myocardial performance index and biochemical markers for early detection of anthracycline-induced cardiotoxicity in adults. Clinical research in cardiology: official journal of the German Cardiac Society 2008; 97:318-26.
17. Fallah-Rad N, Walker J R, Wassef A, et al. The utility of cardiac biomarkers, tissue velocity and strain imaging, and cardiac magnetic resonance imaging in predicting early left ventricular dysfunction in patients with human epidermal growth factor receptor II-positive breast cancer treated with adjuvant trastuzumab therapy. J Am Coll Cardiol 2011; 57:2263-70.
18. Ky B, Putt M, Sawaya H, et al. Early increase in multiple biomarkers predict subsequent cardiotoxicity in patients with breast cancer treated with doxorubicin, taxanes, and trastuzumab. J Am Coll Cardiol 2014; 63:809-16.
19. Timolati F, Ott D, Pentassuglia L, et al. Neuregulin-1 beta attenuates doxorubicin-induced alterations of excitation-contraction coupling and reduces oxidative stress in adult rat cardiomyocytes. Journal of molecular and cellular cardiology 2006; 41:845-54.
20. Kang Y J, Chen Y, Epstein P N. Suppression of doxorubicin cardiotoxicity by overexpression of catalase in the heart of transgenic mice. The Journal of biological chemistry 1996; 271:12610-6.
21. Terwoord J D, Beyer A M, Gutterman D D. Endothelial dysfunction as a complication of anti-cancer therapy. Pharmacol Ther 2022; 237:108116.
22. Luu A Z, Chowdhury B, Al-Omran M, Teoh H, Hess D A, Verma S. Role of Endothelium in Doxorubicin-Induced Cardiomyopathy. JACC Basic Transl Sci 2018; 3:861-70.
23. Grakova E V, Shilov S N, Kopeva K V, et al. Anthracycline-Induced Cardiotoxicity: The Role of Endothelial Dysfunction. Cardiology 2021; 146:315-23.
24. Todorova V K, Hsu P C, Wei J Y, et al. Biomarkers of inflammation, hypercoagulability and endothelial injury predict early asymptomatic doxorubicin-induced cardiotoxicity in breast cancer patients. Am J Cancer Res 2020; 10:2933-45.
25. Ching C, Gustafson D, Thavendiranathan P, Fish J E. Cancer therapy-related cardiac dysfunction: is endothelial dysfunction at the heart of the matter? Clin Sci (Lond) 2021; 135:1487-503.
26. Finkelman B S, Putt M, Wang T, et al. Arginine-Nitric Oxide Metabolites and Cardiac Dysfunction in Patients With Breast Cancer. J Am Coll Cardiol 2017; 70:152-62.
27. Wilkinson E L, Sidaway J E, Cross M J. Statin regulated ERK5 stimulates tight junction formation and reduces permeability in human cardiac endothelial cells. J Cell Physiol 2018; 233:186-200.
28. Wilkinson E L, Sidaway J E, Cross M J. Cardiotoxic drugs Herceptin and doxorubicin inhibit cardiac microvascular endothelial cell barrier formation resulting in increased drug permeability. Biol Open 2016; 5:1362-70.
29. Galan-Arriola C, Vilchez-Tschischke J P, Lobo M, et al. Coronary microcirculation damage in anthracycline cardiotoxicity. Cardiovasc Res 2022; 118:531-41.
30. Hoffman R K, Kim B J, Shah P D, Carver J, Ky B, Ryeom S. Damage to cardiac vasculature may be associated with breast cancer treatment-induced cardiotoxicity. Cardiooncology 2021; 7:15.
31. Kastora S L, Pana T A, Sarwar Y, Myint P K, Mamas M A. Biomarker Determinants of Early Anthracycline-Induced Left Ventricular Dysfunction in Breast Cancer: A Systematic Review and Meta-Analysis. Mol Diagn Ther 2022; 26:369-82.
32. Xiao H, Wang X, Li S, Liu Y, Cui Y, Deng X. Advances in Biomarkers for Detecting Early Cancer Treatment-Related Cardiac Dysfunction. Front Cardiovasc Med 2021; 8:753313.
33. Szczepaniak P, Siedlinski M, Hodorowicz-Zaniewska D, et al. Breast cancer chemotherapy induces vascular dysfunction and hypertension through NOX4 dependent mechanism. The Journal of Clinical Investigation 2022.
34. Demissei B G, Hubbard R A, Zhang L, et al. Changes in Cardiovascular Biomarkers With Breast Cancer Therapy and Associations With Cardiac Dysfunction. J Am Heart Assoc 2020; 9:e014708.
35. Liu D, Ma Z, Yang J, et al. Prevalence and prognosis significance of cardiovascular disease in cancer patients: a population-based study. Aging (Albany NY) 2019; 11:7948-60.
36. Gui H, She R, Luzum J, et al. Plasma Proteomic Profile Predicts Survival in Heart Failure with Reduced Ejection Fraction. Circulation: Genomic and Precision Medicine 2021; 14:e003140.
37. Blanco-Dominguez R, Sanchez-Diaz R, de la Fuente H, et al. A novel circulating microRNA for the detection of acute myocarditis. New England Journal of Medicine 2021; 384:2014-27.
38. Blaser M C, Kraler S, Luscher T F, Aikawa E. Multi-omics approaches to define calcific aortic valve disease pathogenesis. Circulation Research 2021; 128:1371-97.
39. Gustafson D, Ngai M, Wu R, et al. Cardiovascular signatures of COVID-19 predict mortality and identify barrier stabilizing therapies. EBioMedicine 2022; 78:103982.
40. Langley R J, Tsalik E L, Velkinburgh JCv, et al. An Integrated Clinico-Metabolomic Model Improves Prediction of Death in Sepsis. Science Translational Medicine 2013; 5:195ra95-ra95.
41. Govaere O, Cockell S, Tiniakos D, et al. Transcriptomic profiling across the nonalcoholic fatty liver disease spectrum reveals gene signatures for steatohepatitis and fibrosis. Science Translational Medicine 2020; 12:eaba4448.
42. Chao H Y, Wu C C, Singh A, et al. Using Machine Learning to Develop and Validate an In-Hospital Mortality Prediction Model for Patients with Suspected Sepsis. Biomedicines 2022; 10.
43. Ambale-Venkatesh B, Yang X, Wu C O, et al. Cardiovascular Event Prediction by Machine Learning: The Multi-Ethnic Study of Atherosclerosis. Circ Res 2017; 121:1092-101.
44. Houbois C P, Nolan M, Somerset E, et al. Serial Cardiovascular Magnetic Resonance Strain Measurements to Identify Cardiotoxicity in Breast Cancer: Comparison With Echocardiography. JACC Cardiovasc Imaging 2021; 14:962-74.
45. Bloom M W, Hamo C E, Cardinale D, et al. Cancer therapy-related cardiac dysfunction and heart failure: part 1: definitions, pathophysiology, risk factors, and imaging. Circulation: Heart Failure 2016; 9:e002661.
46. Association W M. World Medical Association Declaration of Helsinki: ethical principles for medical research involving human subjects. Jama 2013; 310:2191-4.
47. Lacroix R, Judicone C, Mooberry M, Boucekine M, Key N S, Dignat-George F. Standardization of pre-analytical variables in plasma microparticle determination: results of the International Society on Thrombosis and Haemostasis SSC Collaborative workshop. Journal of thrombosis and haemostasis: JTH 2013.
48. Love M I, Huber W, Anders S. Moderated estimation of fold change and dispersion for RNA-seq data with DESeq2. Genome biology 2014; 15:1-21.
49. Maza E, Frasse P, Senin P, Bouzayen M, Zouine M. Comparison of normalization methods for differential gene expression analysis in RNA-Seq experiments: A matter of relative size of studied transcriptomes. Commun Integr Biol 2013; 6:e25849.
50. BioCarta. Biotech Software & Internet Report 2001; 2:117-20.
51. Kanehisa M, Goto S. KEGG: kyoto encyclopedia of genes and genomes. Nucleic Acids Res 2000; 28:27-30.
52. Jassal B, Matthews L, Viteri G, et al. The reactome pathway knowledgebase. Nucleic Acids Res 2020; 48:D498-d503.
53. Sales A R K, Negrao M V, Testa L, et al. Chemotherapy acutely impairs neurovascular and hemodynamic responses in women with breast cancer. Am J Physiol Heart Circ Physiol 2019; 317:H1-H12.
54. Hader S N, Zinkevich N, Norwood Toro L E, et al. Detrimental effects of chemotherapy on human coronary microvascular function. Am J Physiol Heart Circ Physiol 2019; 317:H705-H10.
55. Saiki H, Moulay G, Guenzel A J, et al. Experimental cardiac radiation exposure induces ventricular diastolic dysfunction with preserved ejection fraction. Am J Physiol Heart Circ Physiol 2017; 313:H392-H407.
56. Li X, Gu J, Zhang Y, et al. 1-arginine alleviates doxorubicin-induced endothelium-dependent dysfunction by promoting nitric oxide generation and inhibiting apoptosis. Toxicology 2019; 423:105-11.
57. Todorova V K, Wei J Y, Makhoul I. Subclinical doxorubicin-induced cardiotoxicity update: role of neutrophils and endothelium. Am J Cancer Res 2021; 11:4070-91.
58. Chow A Y, Chin C, Dahl G, Rosenthal D N. Anthracyclines cause endothelial injury in pediatric cancer patients: a pilot study. J Clin Oncol 2006; 24:925-8.
59. Cedervall J, Herre M, Dragomir A, et al. Neutrophil extracellular traps promote cancer-associated inflammation and myocardial stress. Oncoimmunology 2022; 11:2049487.
60. Pavo N, Raderer M, Hulsmann M, et al. Cardiovascular biomarkers in patients with cancer and their association with all-cause mortality. Heart 2015; 101:1874-80.
61. Aguilar-Cazares D, Chavez-Dominguez R, Marroquin-Mucino M, et al. The systemic-level repercussions of cancer-associated inflammation mediators produced in the tumor microenvironment. Front Endocrinol (Lausanne) 2022; 13:929572.
62. Kostner A H, Nielsen P S, Georgsen J B, et al. Systemic Inflammation Associates With a Myeloid Inflamed Tumor Microenvironment in Primary Resected Colon Cancer-May Cold Tumors 20 Simply Be Too Hot? Front Immunol 2021; 12:716342.
63. Marchant D J, Boyd J H, Lin D C, Granville D J, Garmaroudi F S, McManus B M. Inflammation in myocardial diseases. Circ Res 2012; 110:126-44.
64. Harrington J, Nixon A B, Daubert M A, et al. Circulating Angiokines Are Associated With Reverse Remodeling and Outcomes in Chronic Heart Failure. J Card Fail 2023.
65. Eleuteri E, Di Stefano A, Giordano A, et al. Prognostic value of angiopoietin-2 in patients with chronic heart failure. Int J Cardiol 2016; 212:364-8.
66. Peplinski B S, Houston B A, Bluemke D A, et al. Associations of Angiopoietins With Heart Failure Incidence and Severity. J Card Fail 2021; 27:786-95.
67. Bersell K, Arab S, Haring B, Kuhn B. Neuregulin1/ErbB4 signaling induces cardiomyocyte proliferation and repair of heart injury. Cell 2009; 138:257-70.
68. Negishi T et al JACC Cardiovasc Imaging 2018 August; 11(8):1098-1105.DOI: 10.1016/j.jcmg.2018.03.019).
69. Thaevendiranathan P et al, Eur Heart J Cardiovasc Pharmacother 2023 April 29; 9(6):515-525. doi: 10.1093/ehjcvp/pvad031

1. A method of assaying a sample in a subject with cancer prescribed or receiving an anthracycline and/or HER2 targeted therapy cancer treatment, comprising:
a) obtaining a blood sample from the subject with cancer receiving the anthracycline and/or HER2 targeted therapy cancer treatment;
b) detecting or measuring levels of each of a set of biomarkers in the sample obtained from the subject with cancer, the set of biomarkers comprising at least one of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2;
ANGPT2 and ENG; ANGPT2 and ET-1; Endoglin and MPO; ANGPT2, MPO and Endoglin; or ANGPT2, MPO, Endoglin, ET-1; or
a) obtaining a blood sample from the subject with cancer;
b) detecting or measuring levels of each of a set of biomarkers in the sample obtained from the subject with cancer, the set of biomarkers comprising at least one of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2; ANGPT2 and ENG; ANGPT2 and ET-1; Endoglin and MPO; ANGPT2, MPO and Endoglin; or ANGPT2, MPO, Endoglin, ET-1; and
c) identifying the subject as being at an increased or decreased risk of developing CTRCD based on the presence of or the measured level of the set of biomarkers.

2.-7. (canceled)

8. The method of claim 1, wherein the set of biomarkers comprises or consists of MPO, ANGPT2, ENG and ET-1; wherein the cancer is selected from a breast cancer, thymoma, sarcoma, a gastrointestinal cancer, a hematological cancer, lung cancer, renal cancer or melanoma and/or the blood sample is a plasma or serum sample.

9.-10. (canceled)

11. The method of claim 9, wherein the breast cancer is human epidermal growth factor 2 positive (HER2+) breast cancer, optionally wherein the HER2+ breast cancer is stage I, II, or III HER2+ breast cancer; and/or wherein the sample obtained from the subject is obtained prior to starting the cancer treatment.

12.-13. (canceled)

14. The method of claim 11, wherein the set of biomarkers measured or detected is ANGPT2, ENG, ET-1 and MPO.

15. The method of claim 1, wherein the subject has initiated the cancer treatment, wherein the sample is obtained within 6 months of diagnosis or initiation of the cancer treatment and/or wherein the cancer treatment further comprises radiation therapy.

16.-18. (canceled)

19. The method of claim 11, wherein the cancer treatment is an anthracycline and a Her-2 specific monoclonal antibody, optionally a humanized monoclonal antibody; or wherein the cancer treatment comprises a HER2 targeted therapy and the HER2 targeted therapy comprises trastuzumab, pertuzumab, or tucatinib or analogs of any thereof or combinations of any thereof.

20. The method of claim 1, wherein the anthracycline is selected from daunorubicin, doxorubicin, epirubicin, idarubicin, mitoxantrone or valrubicin or an analogue of any thereof or combinations of any thereof; wherein the anthracycline is doxorubicin or a doxorubicin analogue, optionally daunorubicin, epirubicin and idarubicin; wherein the sample was obtained after initiation of anthracycline treatment and the set of biomarkers comprises ANGPT2, E-Selectin and Endoglin; wherein the sample was obtained after initiation of HER2 targeted therapy and the set of biomarkers comprises ANGPT2, E-Selectin, Endoglin and ET-1 or wherein the subject is receiving a HER2 targeted therapy and the sample was obtained from the subject within 3 months of initiating the HER2 targeted therapy treatment.

21.-28. (canceled)

29. The method of claim 1, further comprising detecting a presence of or measuring a level of one or more miRNAs, optionally wherein the one or more miRNAs comprises miR-6727-5P and/or miR-1915-3P and/or or more miRNAs comprise any of the miRNAs in FIG. 5; detecting or measuring one or more clinical parameter identified in FIG. 5; obtaining cardiac clinical data and/or imaging data, optionally the imaging data comprises one or more of cardiac magnetic resonance imaging (MRI) and echocardiography, optionally the MRI left ventricular global longitudinal and circumferential strain; one or more clinical variable as identified in Supplemental Table III and/or wherein the identifying step comprises comparing the measured level of the one or more biomarkers, the measured level of one or more miRNAs, the cardiac clinical data and/or imaging data, and/or the one or more clinical variables to a reference level, profile or score, optionally via a trained classifier.

30.-37. (canceled)

38. A method for treating a subject who is identified as being at risk of developing cancer therapy-related cardiac dysfunction (CTRCD) comprising:
a) detecting or measuring levels of a set of biomarkers in a sample obtained from a subject with cancer, optionally selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α;
b) identifying the subject as being at risk of developing CTRCD based on the levels of the set of biomarkers; and
c) administering a cardioprotective therapy and/or modifying the cancer therapy.

39. A method of identifying if a subject with cancer to be treated with or an anthracycline and/or a HER2 targeted therapy treatment is likely to benefit from a cardioprotective treatment, the method comprising,
a) detecting or measuring levels of each of a set of biomarkers associated with endothelial activation/dysfunction and/or inflammation in a sample obtained from the subject with cancer, the set of biomarkers comprising at least two of angiopoietin-2 (ANGPT2), Endoglin (ENG), E-selectin, endothelin-1 (ET-1), myeloperoxidase (MPO), Interferon gamma-induced protein-10 (IP-10) and Interferon-α, optionally MPO and ANGPT2; ANGPT2 and ENG; E-selectin and ET-1; ANGPT2, E-Selectin and Endoglin; or ANGPT2, E-Selectin, Endoglin, ET-1;
wherein the subject is likely to benefit when the levels of the set of biomarkers is indicative of an increased risk of CTRCD.

40.-46. (canceled)

47. The method of claim 38, wherein the set of biomarkers comprises or consists of MPO, ANGPT2 and ENG or MPO, ANGPT2, ENG and ET-1; wherein the cancer is selected from a breast cancer, thymoma, sarcoma, a gastrointestinal cancer, a hematological cancer, lung cancer, renal cancer or melanoma; and/or the blood sample is a plasma or serum sample.

48. (canceled)

49. The method of claim 47, wherein the breast cancer is human epidermal growth factor 2 positive (HER2+) breast cancer, optionally wherein the HER2+ breast cancer is stage I, II, or III HER2+ breast cancer; and/or wherein the sample obtained from the subject is obtained prior to starting the cancer treatment.

50.-52. (canceled)

53. The method of claim 38, wherein the subject has initiated the cancer treatment, wherein the sample is obtained within 6 months of diagnosis or initiation of the cancer treatment and/or wherein the cancer treatment further comprises radiation therapy.

54.-55. (canceled)

56. The method of claim 53, wherein the set of biomarkers measured or detected is E-selectin and/or ET-1, wherein an increased level in the sample obtained early in the cancer treatment compared to a control (e.g., pre-treatment control) is indicative of developing CTRCD.

57. The method of claim 53, wherein the cancer treatment is an anthracycline and a HER2 specific monoclonal antibody, optionally a humanized monoclonal antibody or wherein the cancer treatment comprises a HER2 targeted therapy and the HER2 targeted therapy comprises trastuzumab, pertuzumab, or tucatinib or analogs of any thereof or combinations of any thereof.

58. The method of claim 38, wherein the anthracycline is selected from daunorubicin, doxorubicin, epirubicin, idarubicin, mitoxantrone or valrubicin or an analogue of any thereof or combinations of any thereof; wherein the anthracycline is doxorubicin or a doxorubicin analogue, optionally daunorubicin, epirubicin and idarubicin; wherein the sample was obtained after initiation of anthracycline treatment and the set of biomarkers comprises ANGPT2, E-Selectin and Endoglin; or wherein the sample was obtained after initiation of HER2 targeted therapy and the set of biomarkers comprises ANGPT2, E-Selectin, Endoglin and ET-1.

59.-66. (canceled)

67. The method claim 38, further comprising: detecting a presence of or measuring a level of one or more miRNAs, optionally wherein the one or more miRNAs comprises miR-6727-5P and/or miR-1915-3P and/or or more miRNAs comprise any of the miRNAs in FIG. 5; detecting or measuring one or more clinical parameter identified in FIG. 5; obtaining cardiac clinical data and/or imaging data, optionally the imaging data comprises one or more of cardiac magnetic resonance imaging (MRI) and echocardiography, optionally the MRI left ventricular global longitudinal and circumferential strain; one or more clinical variable as identified in Supplemental Table III and/or wherein the identifying step comprises comparing the measured level of the one or more biomarkers, the measured level of one or more miRNAs, the cardiac clinical data and/or imaging data, and/or the one or more clinical variables to a reference level, profile or score, optionally via a trained classifier.

68.-74. (canceled)

75. The method of claim 38, wherein the blood sample is plasma or serum fraction and/or wherein the method further comprises determining whether the subject has hyperlipidemia.

76. (canceled)

77. A panel or kit optionally for use with the method of claim 38, the panel or kit comprising a plurality of detection agents specific for each of a set of biomarkers in a blood sample obtained from a subject with cancer prescribed or receiving treatment with or an anthracycline and/or a HER2 targeted therapy treatment, the set of biomarkers selected from: angiopoietin-2 (ANGPT2), Endoglin (ENG), E-selectin, endothelin-1 (ET-1), myeloperoxidase (MPO), Interferon gamma-induced protein-10 (IP-10) and Interferon-α, and optionally a solid support.

78.-85. (canceled)

86. The panel or kit of claim 77, wherein the panel or kit is a plate or bead, optionally the panel or kit is an enzyme-linked immunosorbent assay (ELISA); wherein the panel or kit is for detecting cancer therapy-related cardiac dysfunction (CTRCD) risk in a subject; and/or wherein the subject is identified as being at risk of developing CTRCD, the cardioprotective therapy is administered, optionally the cardioprotective therapy is selected from dexrazoxane, dantrolene, statins and SGTL2 inhibitors.

87.-89. (canceled)
